(19) United States
(12) Patent Application Publication
(10) Pub. No.: US 2018/0148648 A1
(43) Pub. Date: May 31, 2018

(54) REACTIVE MESOGENS

(71) Applicant: Merck Patent GmbH, Darmstadt (DE)

(72) Inventors: Graham SMITH, Southampton (GB); Mark GOEBEL, Winchester (GB); Kevin ADLEM, Bournemouth (GB); Sarah WHITEHOUSE, Southampton (GB); Tara PERRETT, Bournemouth (GB)

(73) Assignee: Merck Patent GmbH, Darmstadt (DE)

(21) Appl. No.: 15/575,415

(22) PCT Filed: May 2, 2016
(86) PCT No.: PCT/EP2016/000715

§ 371 Date: Nov. 20, 2017

(30) Foreign Application Priority Data
May 21, 2015 (EP) 15001529.5
May 21, 2015 (GB) 1508746.3

(51) Int. Cl. C09K 19/38; C07C 69/80; C09K 19/04

(52) U.S. Cl. CPC C09K 19/3861; C09K 2019/0448; C07C 69/80; C09K 19/3842

(57) ABSTRACT

The invention relates to lateral fluorinated reactive mesogens (RMs) comprising a tolane group, to mixtures and formulations comprising them, to polymers obtained form such RMs and RM mixtures, and the use of the RMs, RM mixtures and polymers in optical or electrooptical components or devices, like optical films for liquid crystal displays (LCDs).

### FIELD OF THE INVENTION

The invention relates to lateral fluorinated reactive mesogens (RMs) comprising a tolane group, to mixtures and formulations comprising them, to polymers obtained from such RMs and RM mixtures, and the use of the RMs, RM mixtures and polymers in optical or electrooptical components or devices, like optical films for liquid crystal displays (LCDs).

### Background and Prior Art

Reactive mesogens (RMs), mixtures or formulations comprising them, and polymers obtained thereof, can be used to make optical components, like compensation, retardation or polarisation films, or lenses. These optical components can be used in optical or electrooptical devices like LC displays. Usually the RMs or RM mixtures are polymerised through the process of in-situ polymerisation.

The manufacture of RM film products with high birefringence is of high importance for manufacturing optical components of modern display devices like LCDs. For Example, brightness enhancement films such as 3M DBEF™, are often included in displays in order to increase the brightness or reduce the number of light sources in the backlight unit. Broadband cholesteric films can also be used for this purpose, and the optical properties are dependent upon the broadening which can be achieved during processing. Films which are better able to broaden can be processed faster on a production line, and additionally can have improved optical properties.

In this regard, it is possible to polymerise cholesteric reactive mesogen films such that a gradient in the helical pitch is obtained, thereby broadening the reflection band of the film. Thin films with good optical properties are dependent on the inclusion of at least one suitable high birefringence RM.

Broadening of cholesteric films is dictated by the structure of the high birefringence material in the reactive mesogen mixture. Compounds must be highly birefringent and allow band broadening to occur whilst also having good solubility and a broad nematic range, preferably without melting points becoming too high. High birefringence reactive mesogens made to date with these characteristics only allow cholesteric films to be broadened by a certain amount before films become hazy.

Increasing the birefringence of the RM whilst keeping them polymerisable and with good physical properties is possible, but requires the incorporation of specific chemical groups, like for example tolane groups, into the compounds.

Mesogenic tolane derivatives are known for example from U.S. Pat. No. 6,514,578 B1, GB 2 388 599 B1, U.S. Pat. No. 7,597,942 B1, US 2003-072893 A1 and US 2006-0119783 A1.

Generally tolane groups are relatively reactive and are mostly unsuited to light exposure, making them difficult to utilise in many optical applications due to yellowing or other degradation effects. Furthermore, mesogenic tolane derivatives often show a limited solubility in RM mixtures and are therefore limited in their use.

It is therefore an aim of the present invention to provide improved RMs, RM mixtures and RM formulations, which do not have the drawbacks of materials known from prior art. In particular it is an aim to provide RMs and RM mixtures and RM formulations that are suitable for preparing polymers by in situ UV photopolymerisation, and exhibit at the same time a high birefringence, exhibit a good solubility, show an improved broadening potential, have favorable transition temperatures, and show high resistance against yellowing after being exposed to UV light. Other aims of the invention are immediately evident to the expert from the following description.

Surprisingly, the inventors of the present invention have found that the addition of fluoro lateral groups to polymerisable mesogenic tolane compounds has especially increased the broadening potential of these compound class significantly.

### SUMMARY OF THE INVENTION

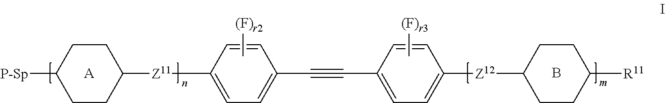 [0012] P is a polymerisable group,
Sp is a spacer group or a single bond,
r1, r2 and r3 are independently of each other 0, 1, 2, 3 or 4, with
r1+r2+r3≥1
R¹¹ is alkyl, alkoxy, thioalkyl, alkylcarbonyl, alkoxycarbonyl, alkylcarbonyloxy or alkoxycarbonyloxy preferably with 1 to 15 C atoms which is more preferably optionally fluorinated,
A and B denote, in case of multiple occurrence independently of one another, an aromatic or alicyclic group, which optionally contains one or more heteroatoms selected from N, O and S, and is optionally substituted by (F)ᵣ₁, preferably 1,4-phenylene, pyridine-2,5-diyl, pyrimidine-2,5-diyl, thiophene-2,5-diyl, naphthalene-2,6-diyl, 1,2,3,4-tetrahydro-naphthalene-2,6-diyl, indane-2,5-diyl, bicyclooctylene or 1,4-cyclohexylene wherein one or two non-adjacent CH₂ groups are optionally replaced by O and/or S, wherein these groups are unsubstituted or substituted by (F)ᵣ₁,
Z¹¹ and Z¹² denotes, in case of multiple occurrence independently of one another, —O—, —S—, —CO—, —COO—, —OCO—, —S—CO—, —CO—S—, —O—COO—, —CO—NR⁰⁰—, —NR⁰⁰—CO—, —NR⁰⁰—CO—NR⁰⁰, —NR⁰⁰—CO—O—, —O—CO—NR⁰⁰—, —OCH₂—, —CH₂O—, —SCH₂—, —CH₂S—, —CF₂O—, —OCF₂—, —CF₂S—, —SCF₂—, —CH₂CH₂—, —(CH₂)ₙ₁, —CF₂CH₂—, —CH₂CF₂—, —CF₂CF₂—, —CH═N—, —N═CH—, —N═N—, —CH═CR⁰⁰—, —CY¹═CY²—, —C≡C—, —CH═CH—COO—, —OCO—CH═CH— or a single bond, preferably —COO—, —OCO—, —C≡C—, or a single bond,
R⁰⁰ and R⁰⁰⁰ independently of each other denote H or alkyl with 1 to 12 C-atoms,
Y¹ and Y² independently of each other denote H, F, Cl or CN,
n is 1, 2, 3 or 4, preferably 1 or 2, most preferably 1,
m is 0, 1, 2, 3 or 4, preferably 0 or 1, most preferably 0,
n1 is an integer from 1 to 10, preferably 1, 2, 3 or 4.

The invention further relates to a mixture, which is hereinafter referred to as “RM mixture”, comprising two or more RMs, at least one of which is a compound of formula I.

The invention further relates to a formulation, which is hereinafter referred to as “RM formulation”, comprising one or more compounds of formula I or an RM mixture as described above and below, and further comprising one or more solvents and/or additives.

The invention further relates to a polymer obtainable by polymerising a compound of formula I or an RM mixture as described above and below, preferably wherein the RMs are aligned, and preferably at a temperature where the RMs or RM mixture exhibit a liquid crystal phase.

The invention further relates to the use of the compounds of formula I, the RM mixture or the polymer as described above and below in optical, electrooptical or electronic components or devices.

The invention further relates to an optical, electrooptical or electronic device or a component thereof, comprising an RM, RM mixture or polymer as described above and below.

Said components include, without limitation, optical retardation films, polarizers, compensators, beam splitters, reflective films, alignment layers, colour filters, antistatic protection sheets, electromagnetic interference protection sheets, polarization controlled lenses for example for autostereoscopic 3D displays, IR reflection films for example for window applications, and lenses for light guides, focusing and optical effects, eg. 3D, holography, telecomms.

Said devices include, without limitation, electrooptical displays, especially LC displays, autostereoscopic 3D displays, organic light emitting diodes (OLEDs), optical data storage devices, and windows.

### BRIEF DESCRIPTION OF THE DRAWINGS

FIG. 1 shows the comparison in the transmission behavior of a polymer film of RM mixtures according to prior art and a polymer film obtained from a RM mixture according to the invention.

FIG. 2 shows the comparison in the yellowing behavior of RM's according to prior art and a RM according to the invention.

FIG. 3 shows the comparison in the transmission behavior of a polymer film of RM mixtures according to prior art and a polymer film obtained from a RM mixture according to the invention.

### DEFINITIONS OF TERMS

As used herein, the term “RM mixture” means a mixture comprising two or more RMs, and optionally comprising further materials.

As used herein, the term “RM formulation” means at least one RM or RM mixture, and one or more other materials added to the at least one RM or RM mixture to provide, or to modify, specific properties of the RM formulation and/or of the at least one RM therein. It will be understood that an RM formulation is also a vehicle for carrying the RM to a substrate to enable the forming of layers or structures thereon. Exemplary materials include, but are not limited to, solvents, polymerisation initiators, surfactants and adhesion promoters, etc. as described in more detail below.

The term “reactive mesogen” (RM) as used herein means a polymerisable mesogenic or liquid crystalline compound, which is preferably a monomeric compound.

The terms “liquid crystal”, “mesogen” and “mesogenic compound” as used herein mean a compound that under suitable conditions of temperature, pressure and concentration can exist as a mesophase or in particular as a LC phase.

The term “mesogenic group” as used herein means a group with the ability to induce liquid crystal (LC) phase behaviour. Mesogenic groups, especially those of the non-amphiphilic type, are usually either calamitic or discotic. The compounds comprising mesogenic groups do not necessarily have to exhibit an LC phase themselves. It is also possible that they show LC phase behaviour only in mixtures with other compounds, or when the mesogenic compounds or the mixtures thereof are polymerised. For the sake of simplicity, the term “liquid crystal” is used hereinafter for both mesogenic and LC materials.

The term “calamitic” as used herein means a rod- or board/lath-shaped compound or group. The term “banana-shaped” as used herein means a bent group in which two, usually calamitic, mesogenic groups are linked through a semi-rigid group in such a way as not to be collinear.

The term “discotic” as used herein means a disc- or sheet-shaped compound or group.

A calamitic mesogenic compound is usually comprising a calamitic, i.e. rod- or lath-shaped, mesogenic group consisting of one or more aromatic or alicyclic groups connected to each other directly or via linkage groups, optionally comprising terminal groups attached to the short ends of the rod, and optionally comprising one or more lateral groups attached to the long sides of the rod, wherein these terminal and lateral groups are usually selected e.g. from carbyl or hydrocarbyl groups, polar groups like halogen, nitro, hydroxy, etc., or polymerisable groups.

A discotic mesogenic compound is usually comprising a discotic, i.e. relatively flat disc- or sheet-shaped mesogenic group consisting for example of one or more condensed aromatic or alicyclic groups, like for example triphenylene, and optionally comprising one or more terminal groups that are attached to the mesogenic group and are selected from the terminal and lateral groups mentioned above.

For an overview of terms and definitions in connection with liquid crystals and mesogens see Pure Appl. Chem. 73(5), 888 (2001) and C. Tschierske, G. PelzI and S. Diele, Angew. Chem. 2004, 116, 6340-6368.

Polymerisable compounds with one polymerisable group are also referred to as “monoreactive” compounds, compounds with two polymerisable groups as “direactive” compounds, and compounds with more than two polymerisable groups as “multireactive” compounds. Compounds without a polymerisable group are also referred to as “non-reactive” compounds.

The term “spacer” or “spacer group” as used herein, also referred to as “Sp” below, is known to the person skilled in the art and is described in the literature, see, for example, Pure Appl. Chem. 73(5), 888 (2001) and C. Tschierske, G. Pelzl, S. Diele, Angew. Chem. 2004, 116, 6340-6368.

Unless stated otherwise, the term “spacer” or “spacer group” above and below denotes a flexible organic group, which in a polymerisable mesogenic compound (“RM”) connects the mesogenic group and the polymerisable group(s).

The term “film” as used herein includes rigid or flexible, self-supporting or free-standing films with mechanical stability, as well as coatings or layers on a supporting substrate or between two substrates. “Thin film” means a film having a thickness in the nanometer or micrometer range, preferably at least 10 nm, very preferably at least 100 nm, and preferably not more than 100 μm, very preferably not more than 10 μm.

The term “hydrocarbyl group” means any monovalent or multivalent organic radical moiety which comprises at least one carbon atom and optionally one or more H atoms, and optionally one or more hetero atoms like for example N, O, S, P, Si, Se, As, Te or Ge. A hydrocarbyl group comprising a chain of 3 or more C atoms may also be linear, branched and/or cyclic, including spiro and/or fused rings.

Throughout the application, the term “aryl and heteroaryl groups” encompass groups, which can be monocyclic or polycyclic, i.e. they can have one ring (such as, for example, phenyl) or two or more rings, which may also be fused (such as, for example, naphthyl) or covalently linked (such as, for example, biphenyl), or contain a combination of fused and linked rings. Heteroaryl groups contain one or more heteroatoms, preferably selected from O, N, S and Se. Particular preference is given to mono-, bi- or tricyclic aryl groups having 6 to 25 C atoms and mono-, bi- or tricyclic heteroaryl groups having 2 to 25 C atoms, which optionally contain fused rings, and which are optionally substituted. Preference is furthermore given to 5, 6 or 7-membered aryl and heteroaryl groups, in which, in addition, one or more CH groups may be replaced by N, S or O in such a way that O atoms and/or S atoms are not linked directly to one another. Preferred aryl groups are, for example, phenyl, biphenyl, terphenyl, [1,1′:3′,1″]¬¬terphenyl-2′-yl, naphthyl, anthracene, binaphthyl, phenanthrene, pyrene, dihydropyrene, chrysene, perylene, tetracene, pentacene, benzopyrene, fluorene, indene, indenofluorene, spirobifluorene, more preferably 1,4-phenylene, 4,4′-biphenylene, 1,4-tephenylene.

Preferred heteroaryl groups are, for example, 5 membered rings, such as pyrrole, pyrazole, imidazole, 1,2,3-triazole, 1,2,4-triazole, tetrazole, furan, thiophene, selenophene, oxazole, isoxazole, 1,2 thiazole, 1,3-thiazole, 1,2,3-oxadiazole, 1,2,4 oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 6 membered rings, such as pyridine, pyridazine, pyrimidine, pyrazine, 1,3,5-triazine, 1,2,4-triazine, 1,2,3-triazine, 1,2,4,5-tetrazine, 1,2,3,4-tetrazine, 1,2,3,5-tetrazine, or condensed groups, such as indole, iso-indole, indolizine, indazole, benzimidazole, benzotriazole, purine, naphthimidazole, phenanthrimidazole, pyridimidazole, pyrazinimidazole, quinoxalinimidazole, benzoxazole, naphthoxazole, anthroxazole, phen-anthroxazole, isoxazole, benzothiazole, benzofuran, isobenzofuran, dibenzofuran, quinoline, isoquinoline, pteridine, benzo-5,6-quinoline, benzo-6,7-quinoline, benzo-7,8-quinoline, benzoisoquinoline, acridine, phenothiazine, phenoxazine, benzopyridazine, benzopyrimidine, quinoxaline, phenazine, naphthyridine, azacarbazole, benzocarboline, phenanthridine, phenanthroline, thieno[2,3b]thiophene, thieno[3,2b]-thiophene, dithienothiophene, isobenzothiophene, dibenzothiophene, benzothiadiazothiophene, or combinations of these groups. The heteroaryl groups may also be substituted by alkyl, alkoxy, thioalkyl, fluorine, fluoroalkyl or further aryl or heteroaryl groups.

In the context of this application, the term “(non-aromatic) alicyclic and heterocyclic groups” encompass both saturated rings, i.e. those that contain exclusively single bonds, and partially unsaturated rings, i.e. those that may also contain multiple bonds. Heterocyclic rings contain one or more heteroatoms, preferably selected from Si, O, N, S and Se. The (non-aromatic) alicyclic and heterocyclic groups can be monocyclic, i.e. contain only one ring (such as, for example, cyclohexane), or polycyclic, i.e. contain a plurality of rings (such as, for example, decahydro-naphthalene or bicyclooctane). Particular preference is given to saturated groups. Preference is furthermore given to mono-, bi- or tricyclic groups having 3 to 25 C atoms, which optionally contain fused rings and that are optionally substituted. Preference is furthermore given to 5-, 6-, 7- or 8-membered carbocyclic groups in which, in addition, one or more C atoms may be replaced by Si and/or one or more CH groups may be replaced by N and/or one or more non-adjacent CH₂ groups may be replaced by —O— and/or —S—. Preferred alicyclic and heterocyclic groups are, for example, 5-membered groups, such as cyclopentane, tetrahydrofuran, tetrahydrothiofuran, pyrrolidine, 6-membered groups, such as cyclohexane, silinane, cyclohexene, tetrahydropyran, tetrahydrothiopyran, 1,3-dioxane, 1,3-dithiane, piperidine, 7-membered groups, such as cycloheptane, and fused groups, such as tetrahydronaphthalene, decahydronaphthalene, indane, bicyclo[1.1.1]-pentane-1,3-diyl, bicyclo[2.2.2]octane-1,4-diyl, spiro[3.3]heptane-2,6-diyl, octahydro-4,7-methanoindane-2,5-diyl, more preferably 1,4-cyclohexylene 4,4′-bicyclohexylene, 3,17-hexadecahydro-cyclopenta[a]phenanthrene, optionally being substituted by one or more identical or different groups L. Especially preferred aryl-, heteroaryl-, alicyclic- and heterocyclic groups are 1,4-phenylene, 4,4′-biphenylene, 1,4-terphenylene, 1,4-cyclohexylene, 4,4′-bicyclohexylene, and 3,17-hexadecahydro-cyclopenta[a]-phenanthrene, optionally being substituted by one or more identical or different groups L.

Preferred substituents (L) of the above-mentioned aryl-, heteroaryl-, alicyclic- and heterocyclic groups are, for example, solubility-promoting groups, such as alkyl or alkoxy and electron-withdrawing groups, such as fluorine, nitro or nitrile. Particularly preferred substituents are, for example, F, Cl, CN, NO₂, CH₃, C₂H₅, OCH₃, OC₂H₅, COCH₃, COC₂H₅, COOCH₃, COOC₂H₅, CF₃, OCF₃, OCHF₂ or OC₂F₅.

Above and below “halogen” denotes F, Cl, Br or I.

Above and below, the terms “alkyl”, “aryl”, “heteroaryl”, etc., also encompass polyvalent groups, for example alkylene, arylene, heteroarylene, etc. The term “aryl” denotes an aromatic carbon group or a group derived there from. The term “heteroaryl” denotes “aryl” in accordance with the above definition containing one or more heteroatoms.

Preferred alkyl groups are, for example, methyl, ethyl, n propyl, isopropyl, n butyl, isobutyl, s butyl, t butyl, 2 methylbutyl, n pentyl, s pentyl, cyclo-pentyl, n hexyl, cyclohexyl, 2 ethylhexyl, n heptyl, cycloheptyl, n octyl, cyclooctyl, n nonyl, n decyl, n undecyl, n dodecyl, dodecanyl, trifluoro-methyl, perfluoro-n-butyl, 2,2,2-trifluoroethyl, perfluorooctyl, perfluoro-hexyl, etc.

Preferred alkoxy groups are, for example, methoxy, ethoxy, 2-methoxy-ethoxy, n-propoxy, i-propoxy, n-butoxy, i-butoxy, s-butoxy, t-butoxy, 2-methylbutoxy, n-pentoxy, n-hexoxy, n-heptoxy, n-octoxy, n-nonoxy, n-decoxy, n-undecoxy, n-dodecoxy.

Preferred alkenyl groups are, for example, ethenyl, propenyl, butenyl, pentenyl, cyclopentenyl, hexenyl, cyclohexenyl, heptenyl, cycloheptenyl, octenyl, cyclooctenyl.

Preferred alkynyl groups are, for example, ethynyl, propynyl, butynyl, pen-tynyl, hexynyl, octynyl.

Preferred amino groups are, for example, dimethylamino, methylamino, methylphenylamino, phenylamino.

The term “chiral” in general is used to describe an object that is non-superimposable on its mirror image.

“Achiral” (non-chiral) objects are objects that are identical to their mirror image.

The terms “chiral nematic” and “cholesteric” are used synonymously in this application, unless explicitly stated otherwise.

The pitch induced by the chiral substance (P0) is in a first approximation inversely proportional to the concentration (c) of the chiral material used.

The constant of proportionality of this relation is called the helical twisting power (HTP) of the chiral substance and defined by equation (4)

HTP≡1/(c·P0)

wherein
c is concentration of the chiral compound.

### DETAILED DESCRIPTION

Preferred compounds of formula I are those selected of formula Ia or Ib

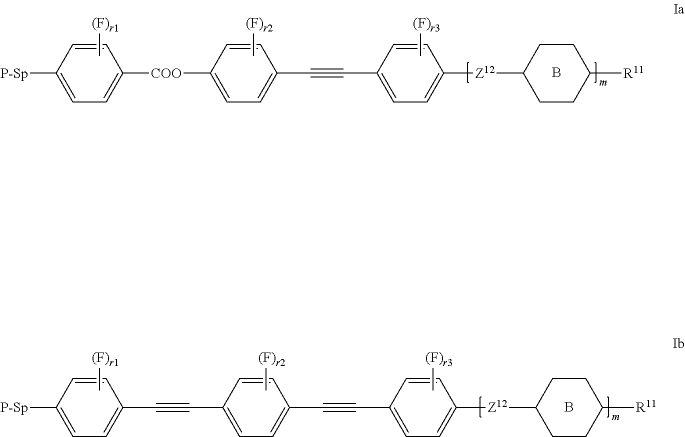

wherein
P is a polymerisable group,
Sp is a spacer group or a single bond,
r1, r2, r3 are independently of each other 0, 1, 2, 3 or 4, with r1+r2+r3≥1, and
R¹¹, Z¹², ring B and m have one of the meanings as given above.

Preferred compounds of formula I are those selected of formula I1 to I6

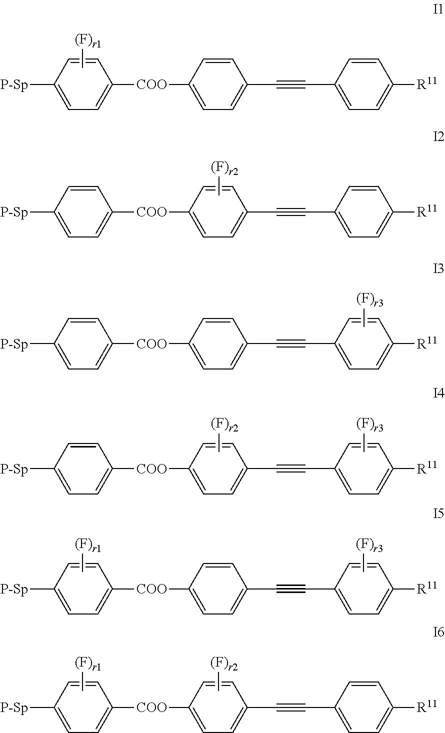

wherein P, Sp, and R¹ are as defined in formula I, r1 to r3 denotes 1, 2, 3, or 4, preferably 1 or 2.

Further preferred are compounds of formula I wherein P is selected from the group consisting of heptadiene, vinyloxy, acrylate, methacrylate, fluoroacrylate, chloroacrylate, oxetane and epoxide groups, and very preferably denotes an acrylate, methacrylate or oxetane group, especially an acrylate or methacrylate group, in particular an acrylate group.

Preferred compounds of formula I1 to I6 are selected of the following formulae

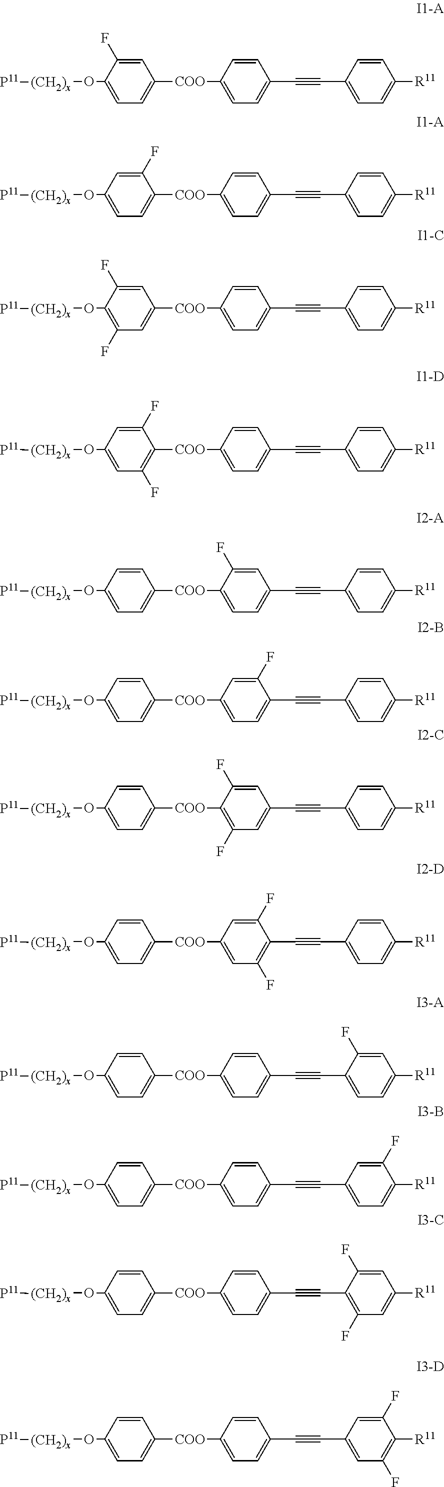

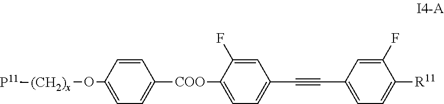

Wherein P¹¹ denotes selected from the group consisting of heptadiene, vinyloxy, acrylate, methacrylate, fluoroacrylate, chloroacrylate, oxetane and epoxide groups, and very preferably denotes an acrylate, methacrylate or oxetane group, especially an acrylate or methacrylate group, in particular an acrylate group, and x is an integer from 0 to 12, preferably from 1 to 8, more preferably 3, 4, 5 or 6, in particular x denotes 3 or 6, especially 6. R¹¹ has one of the meanings as given above under formula I.

Especially preferred are the compounds of formula I2, which are preferably selected from the following formulae:

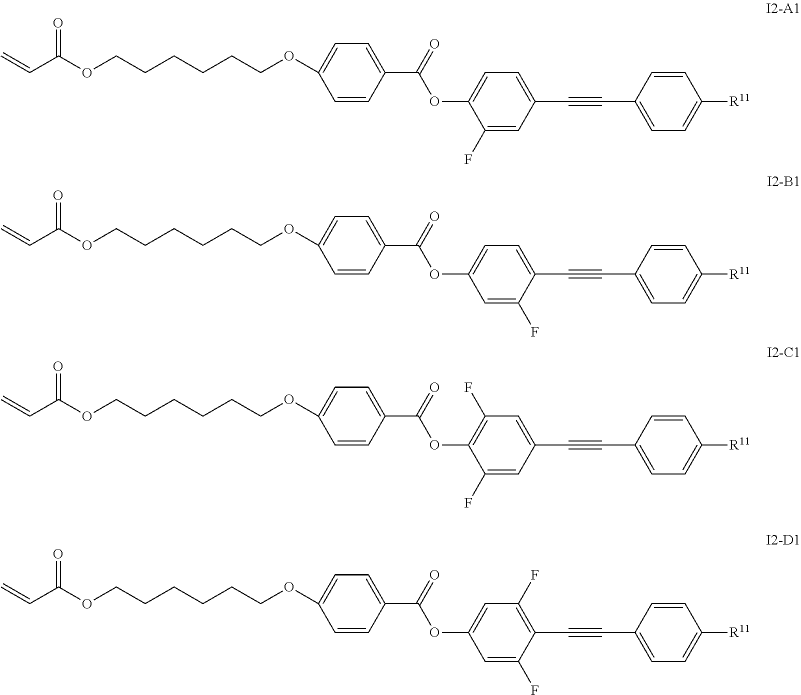

Wherein R¹¹ has one of the meanings as given above under formula I. preferably R¹¹ denotes alkyl or alkoxy.

Further preferred are compounds of formula I2-A1, which are selected from compounds of the following formulae,

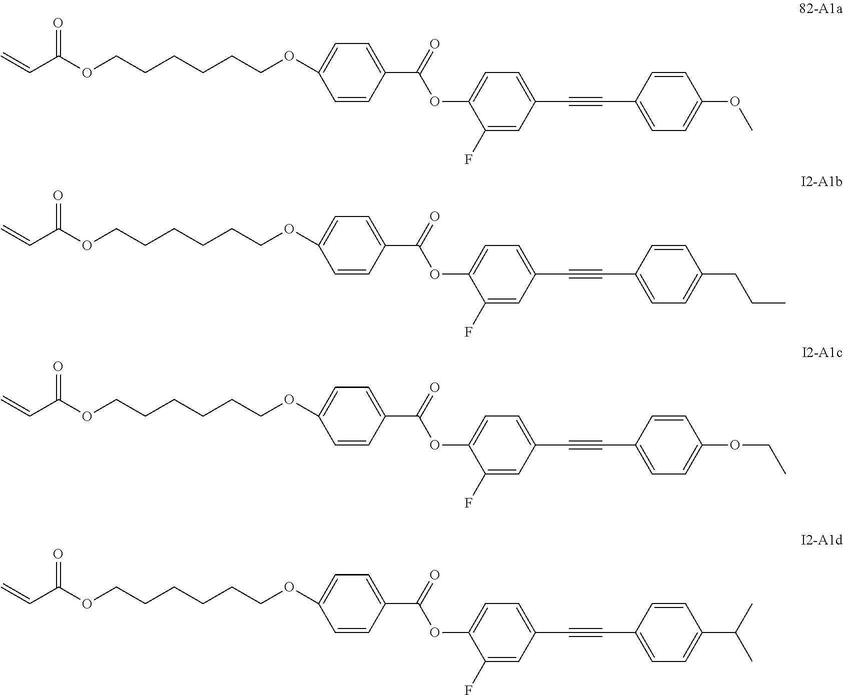

The synthesis of the compounds of formula I and its subformulae can be carried out analogously to the illustrative reactions shown below or in the examples. The preparation of further compounds according to the invention can also be carried out by other methods known per se to the person skilled in the art from the literature.

Exemplarily, the compounds of formula I can be synthesized according to or in analogy to the methods as illustrated in Scheme 1.

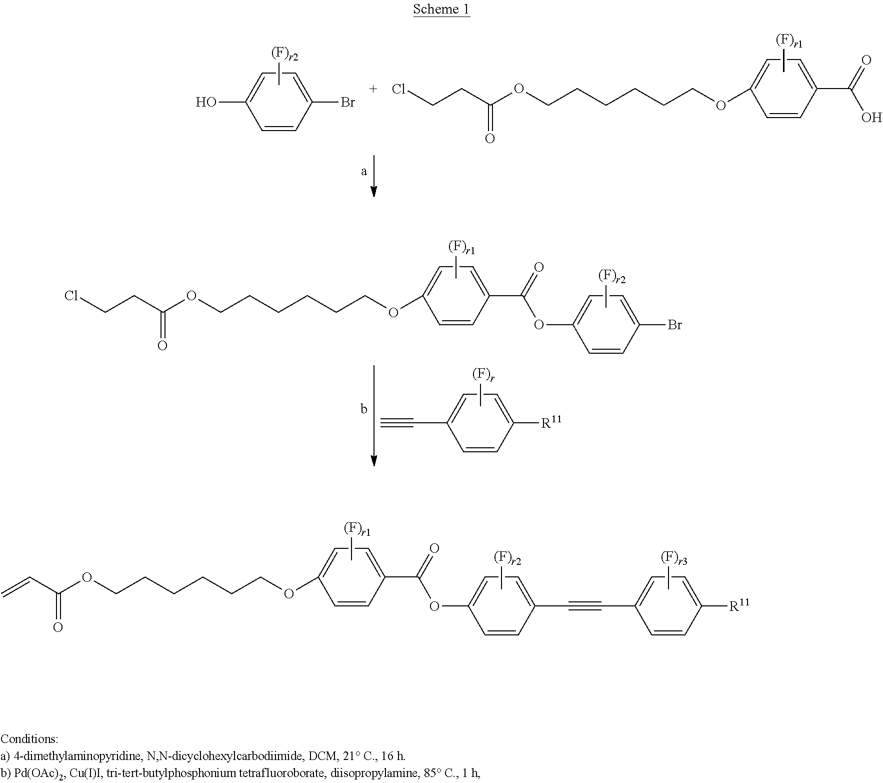

and wherein the parameter R¹¹ and r1 to r3 have one of the meanings as given in formula I.

Another object of the invention is an RM mixture comprising two or more RMs, at least one of which is a compound of formula I.

Preferably the RM mixture comprises one or more RMs having only one polymerisable functional group (monoreactive RMs), at least one of which is a compound of formula I, and one or more RMs having two or more polymerisable functional groups (di- or multireactive RMs).

The di- or multireactive RMs are preferably selected of formula DRM

P¹-Sp¹-MG-Sp²-P² DRM

wherein
P¹ and P² independently of each other denote a polymerisable group,
Sp¹ and Sp² independently of each other are a spacer group or a single bond, and
MG is a rod-shaped mesogenic group, which is preferably selected of formula MG

-(A¹-Z¹)ₙ-A²- MG

wherein
A¹ and A² denote, in case of multiple occurrence independently of one another, an aromatic or alicyclic group, which optionally contains one or more heteroatoms selected from N, O and S, and is optionally mono- or polysubstituted by L,
L is P-Sp-, F, Cl, Br, I, —CN, —NO₂, —NCO, —NCS, —OCN, —SCN, —C(═O)NR^{x}R^{y}, —C(═O)OR^{x}, —C(═O)R^{x}, —NR^{x}R^{y}, —OH, —SF₅, optionally substituted silyl, aryl or heteroaryl with 1 to 12, preferably 1 to 6 C atoms, and straight chain or branched alkyl, alkoxy, alkylcarbonyl, alkoxycarbonyl, alkylcarbonyloxy or alkoxycarbonyloxy with 1 to 12, preferably 1 to 6 C atoms, wherein one or more H atoms are optionally replaced by F or Cl,
R^{x} and R^{y} independently of each other denote H or alkyl with 1 to 12 C-atoms,
Z¹ denotes, in case of multiple occurrence independently of one another, —O—, —S—, —CO—, —COO—, —OCO—, —S—CO—, —CO—S—, —O—COO—, —CO—NR⁰⁰—, —NR⁰⁰—CO—, —NR⁰⁰—CO—NR⁰⁰⁰, —NR⁰⁰—CO—O—, —O—CO—NR⁰⁰—, —OCH₂—, —CH₂O—, —SCH₂—, —CH₂S—, —CF₂O—, —OCF₂—, —CF₂S—, —SCF₂—, —CH₂CH₂—, —(CH₂)ₙ₁, —CF₂CH₂—, —CH₂CF₂—, —CF₂CF₂—, —CH═N—, —N═CH—, —N═N—, —CH═CR⁰⁰—CY¹═CY²—, —C≡C—, —CH═CH—COO—, —OCO—CH═CH— or a single bond, preferably —COO—, —OCO— or a single bond,
R⁰⁰ and R⁰⁰⁰ independently of each other denote H or alkyl with 1 to 12 C-atoms,
Y¹ and Y² independently of each other denote H, F, Cl or CN,
n is 1, 2, 3 or 4, preferably 1 or 2, most preferably 2,
n1 is an integer from 1 to 10, preferably 1, 2, 3 or 4.

Preferred groups A¹ and A² include, without limitation, furan, pyrrol, thiophene, oxazole, thiazole, thiadiazole, imidazole, phenylene, cyclohexylene, bicyclooctylene, cyclohexenylene, pyridine, pyrimidine, pyrazine, azulene, indane, fluorene, naphthalene, tetrahydronaphthalene, anthracene, phenanthrene and dithienothiophene, all of which are unsubstituted or substituted by 1, 2, 3 or 4 groups L as defined above.

Particular preferred groups A¹ and A² are selected from 1,4-phenylene, pyridine-2,5-diyl, pyrimidine-2,5-diyl, thiophene-2,5-diyl, naphthalene-2,6-diyl, 1,2,3,4-tetrahydro-naphthalene-2,6-diyl, indane-2,5-diyl, bicyclooctylene or 1,4-cyclohexylene wherein one or two non-adjacent CH₂ groups are optionally replaced by O and/or S, wherein these groups are unsubstituted or substituted by 1, 2, 3 or 4 groups L as defined above.

Preferred RMs of formula DRM are selected of formula DRMa

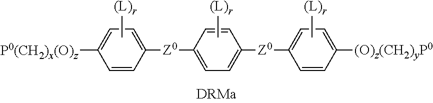

wherein
P⁰ is, in case of multiple occurrence independently of one another, a polymerisable group, preferably an acryl, methacryl, oxetane, epoxy, vinyl, heptadiene, vinyloxy, propenyl ether or styrene group,
Z⁰ is —COO—, —OCO—, —CH₂CH₂—, —CF₂O—, —OCF₂—, —C≡C—, —CH═CH—, —OCO—CH═CH—, —CH═CH—COO—, or a single bond,
L has on each occurrence identically or differently one of the meanings given for L¹ in formula I, and is preferably, in case of multiple occurrence independently of one another, selected from F, Cl, CN or optionally halogenated alkyl, alkoxy, alkylcarbonyl, alkoxycarbonyl, alkylcarbonyloxy or alkoxycarbonyloxy with 1 to 5 C atoms,
r is 0, 1, 2, 3 or 4,
x and y are independently of each other 0 or identical or different integers from 1 to 12,
z is 0 or 1, with z being 0 if the adjacent x or y is 0.

Very preferred RMs of formula DRM are selected from the following formulae:

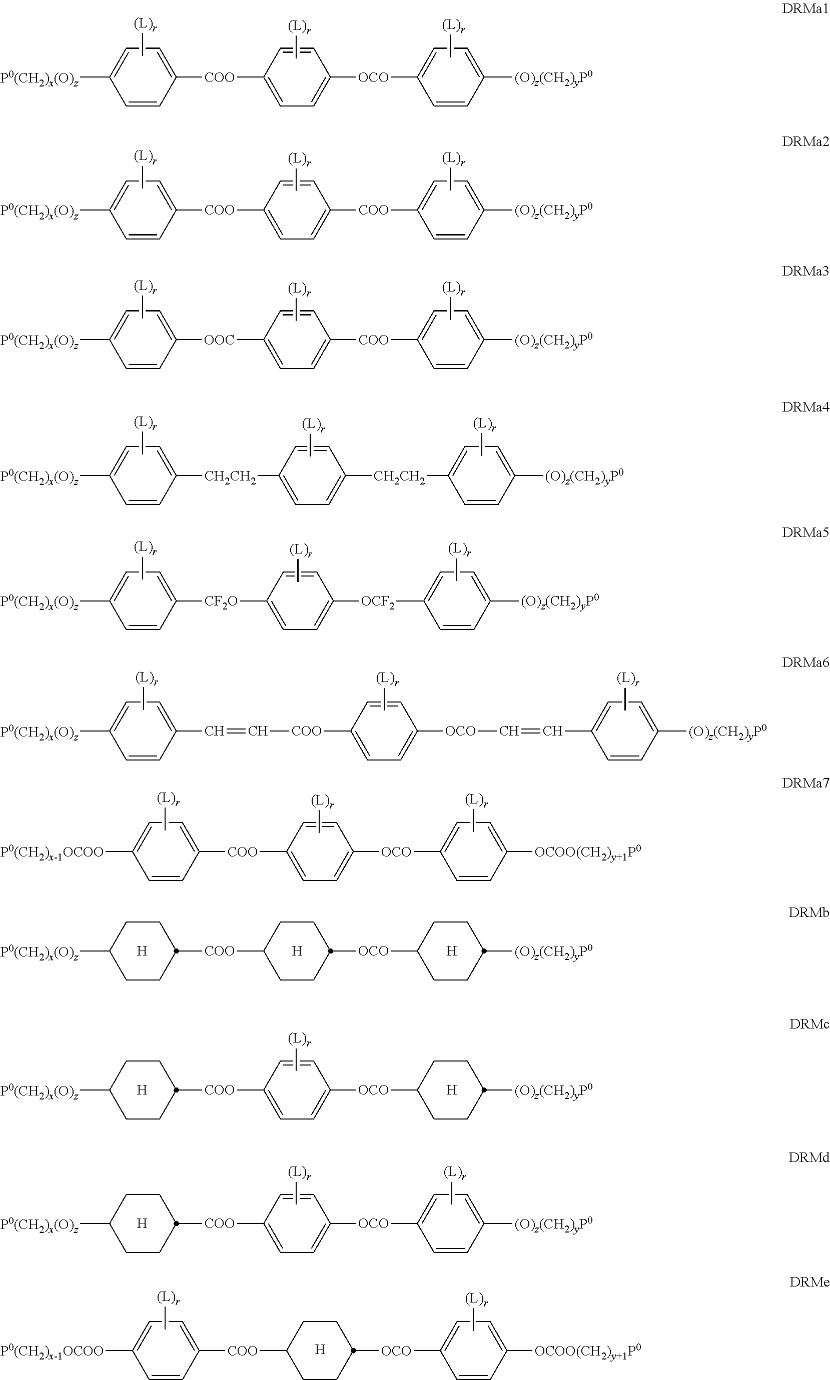

wherein P⁰, L, r, x, y and z are as defined in formula DRMa.

Especially preferred are compounds of formula DRMa1, DRMa2 and DRMa3, in particular those of formula DRMa1.

The concentration of di- or multireactive RMs, preferably those of formula DRM and its subformulae, in the RM mixture is preferably from 1% to 60%, very preferably from 5 to 40%.

In another preferred embodiment the RM mixture comprises, in addition to the compounds of formula I, one or more monoreactive RMs. These additional monoreactive RMs are preferably selected from formula MRM:

P¹—Sp¹-MG-R MRM

wherein P¹, Sp¹ and MG have the meanings given in formula DRM,
R denotes P-Sp-, F, Cl, Br, I, —CN, —NO₂, —NCO, —NCS, —OCN, —SCN, —C(═O)NR^{x}R^{y}, —C(═O)X, —C(═O)OR^{x}, —C(═O)R^{y}, —NR^{x}R^{y}, —OH, —SF₅, optionally substituted silyl, straight chain or branched alkyl, alkoxy, alkylcarbonyl, alkoxycarbonyl, alkylcarbonyloxy or alkoxycarbonyloxy with 1 to 12, preferably 1 to 6 C atoms, wherein one or more H atoms are optionally replaced by F or Cl,
X is halogen, preferably F or Cl, and
R^{x} and R^{y} are independently of each other H or alkyl with 1 to 12 C-atoms.

Preferably the RMs of formula MRM are selected from the following formulae.

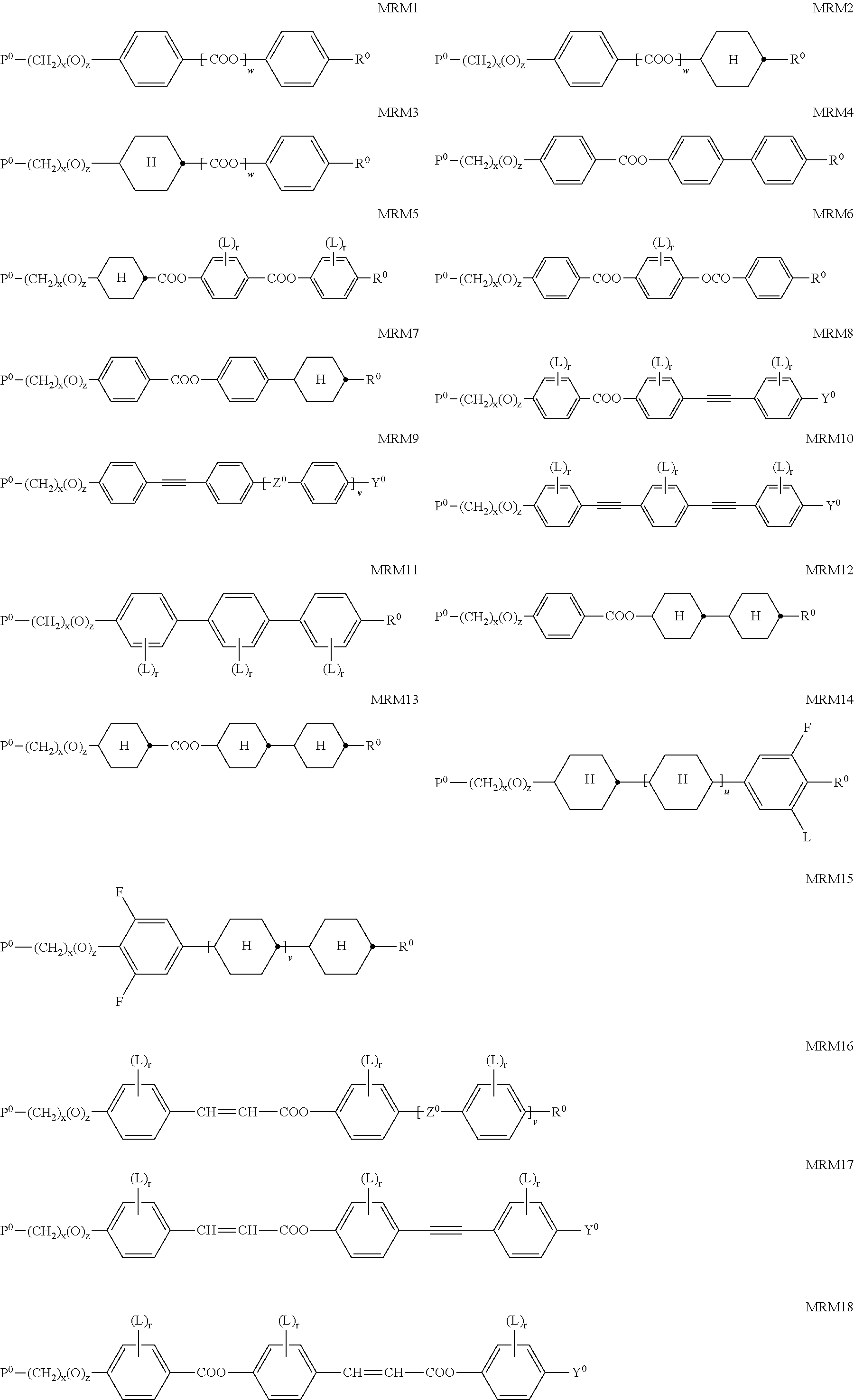

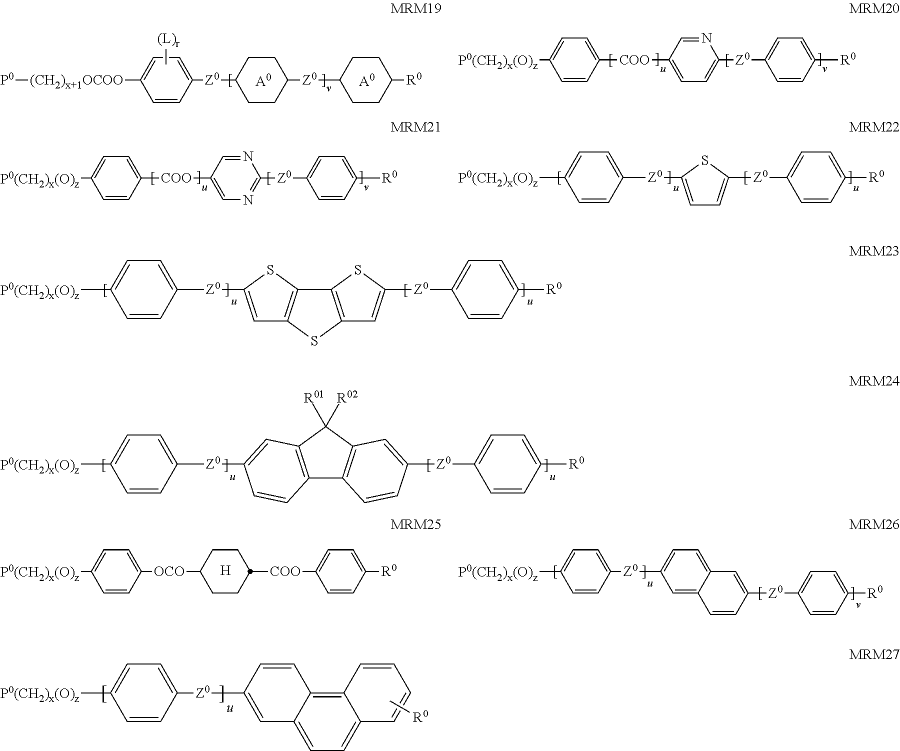

wherein P⁰, L, r, x, y and z are as defined in formula DRMa,
R⁰ is alkyl, alkoxy, thioalkyl, alkylcarbonyl, alkoxycarbonyl, alkylcarbonyloxy or alkoxycarbonyloxy with 1 or more, preferably 1 to 15 C atoms or denotes Y⁰ or P—(CH₂)_{y}—(O)_{z}—,
X⁰ is —O—, —S—, —CO—, —COO—, —OCO—, —O—COO—, —CO—NR⁰¹—, —NR⁰¹—CO—, —NR⁰¹—CO—NR¹—, —OCH₂—, —CH₂O—, —SCH₂—, —CH₂S—, —CF₂O—, —OCF₂—, —CF₂S—, —SCF₂—, —CF₂CH₂—, —CH₂CF₂—, —CF₂CF₂—, —CH═N—, —N═CH—, —N═N—, —CH═CR⁰¹—, —CF═CF—, —C≡C—, —CH═CH—COO—, —OCO—CH═CH— or a single bond
Y⁰ is F, Cl, CN, NO₂, OCH₃, OCN, SCN, SF₅, or mono-oligo- or polyfluorinated alkyl or alkoxy with 1 to 4 C atoms,
Z⁰ is —COO—, —OCO—, —CH₂CH₂—, —CF₂O—, —OCF₂—, —CH═CH—, —OCO—CH═CH—, —CH═CH—COO—, or a single bond,
A⁰ is, in case of multiple occurrence independently of one another, 1,4-phenylene that is unsubstituted or substituted with 1, 2, 3 or 4 groups L, or trans-1,4-cyclohexylene,
R^{01,02} are independently of each other H, R⁰ or Y⁰,
u and v are independently of each other 0, 1 or 2,
w is 0 or 1,
   and wherein the benzene and naphthalene rings can additionally be substituted with one or more identical or different groups L.

Especially preferred are compounds of formula MRM1, MRM2, MRM3, MRM4, MRM5, MRM6, MRM7, MRM9 and MRM10, in particular those of formula MRM1, MRM4, MRM6, and MRM7.

The concentration of all monoreactive RMs, including those of formula I, in the RM mixture is preferably from 1 to 80%, very preferably from 5 to 20%.

The RM mixture preferably exhibits a nematic LC phase, or a smectic LC phase and a nematic LC phase, very preferably a nematic LC phase at room temperature.

In formulae DRM, MRM and their preferred subformulae, L is preferably selected from F, Cl, CN, NO₂ or straight chain or branched alkyl, alkoxy, alkylcarbonyl, alkoxycarbonyl, alkylcarbonlyoxy or alkoxycarbonyloxy with 1 to 12 C atoms, wherein the alkyl groups are optionally perfluorinated, or P-Sp-.

Very preferably L is selected from F, Cl, CN, NO₂, CH₃, C₂H₅, C(CH₃)₃, CH(CH₃)₂, CH₂CH(CH₃)C₂H₅, OCH₃, OC₂H₅, COCH₃, COC₂H₅, COOCH₃, COOC₂H₅, CF₃, OCF₃, OCHF₂, OC₂F₅ or P-Sp-, in particular F, Cl, CN, CH₃, C₂H₅, C(CH₃)₃, CH(CH₃)₂, OCH₃, COCH₃ or OCF₃, most preferably F, Cl, CH₃, C(CH₃)₃, OCH₃ or COCH₃, or P-Sp-.

A substituted benzene ring of the formula

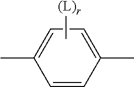

is preferably

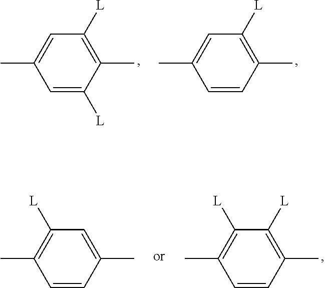

with L having each independently one of the meanings given above.

In formulae I, DRM, MRM and their preferred subformulae, an alkyl or alkoxy radical, i.e. where the terminal CH₂ group is replaced by —O—, can be straight-chain or branched. It is preferably straight-chain, has 2, 3, 4, 5, 6, 7 or 8 carbon atoms and accordingly is preferably ethyl, propyl, butyl, pentyl, hexyl, heptyl, octyl, ethoxy, propoxy, butoxy, pentoxy, hexoxy, heptoxy, or octoxy, furthermore methyl, nonyl, decyl, undecyl, dodecyl, tridecyl, tetradecyl, pentadecyl, nonoxy, decoxy, undecoxy, dodecoxy, tridecoxy or tetradecoxy, for example.

Oxaalkyl, i.e. where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example.

An alkyl group wherein one or more CH₂ groups are replaced by —CH═CH— can be straight-chain or branched. It is preferably straight-chain, has 2 to 10 C atoms and accordingly is preferably vinyl, prop-1-, or prop-2-enyl, but-1-, 2- or but-3-enyl, pent-1-, 2-, 3- or pent-4-enyl, hex-1-, 2-, 3-, 4- or hex-5-enyl, hept-1-, 2-, 3-, 4-, 5- or hept-6-enyl, oct-1-, 2-, 3-, 4-, 5-, 6- or oct-7-enyl, non-1-, 2-, 3-, 4-, 5-, 6-, 7- or non-8-enyl, dec-1-, 2-, 3-, 4-, 5-, 6-, 7-, 8- or dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1 E-alkenyl, C₄-C₇-3E-alkenyl and C₅-C₇-4-alkenyl. Examples for particularly preferred alkenyl groups are vinyl, 1 E-propenyl, 1 E-butenyl, 1E-pentenyl, 1 E-hexenyl, 1 E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Groups having up to 5 C atoms are generally preferred.

In an alkyl group wherein one CH₂ group is replaced by —O— and one by —CO—, these radicals are preferably neighboured. Accordingly these radicals together form a carbonyloxy group —CO—O— or an oxycarbonyl group —O—CO—. Preferably this group is straight-chain and has 2 to 6 C atoms. It is accordingly preferably acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxyethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxy-carbonylmethyl, propoxycarbonylmethyl, butoxycarbonylmethyl, 2-(methoxycarbonyl)ethyl, 2-(ethoxycarbonyl)ethyl, 2-(propoxy-carbonyl)ethyl, 3-(methoxycarbonyl)propyl, 3-(ethoxycarbonyl)propyl, 4-(methoxycarbonyl)-butyl.

An alkyl group wherein two or more CH₂ groups are replaced by —O— and/or —COO— can be straight-chain or branched. It is preferably straight-chain and has 3 to 12 C atoms. Accordingly it is preferably bis-carboxy-methyl, 2,2-bis-carboxy-ethyl, 3,3-bis-carboxy-propyl, 4,4-bis-carboxy-butyl, 5,5-bis-carboxy-pentyl, 6,6-bis-carboxy-hexyl, 7,7-bis-carboxy-heptyl, 8,8-bis-carboxy-octyl, 9,9-bis-carboxy-nonyl, 10,10-bis-carboxy-decyl, bis-(methoxycarbonyl)-methyl, 2,2-bis-(methoxycarbonyl)-ethyl, 3,3-bis-(methoxycarbonyl)-propyl, 4,4-bis-(methoxycarbonyl)-butyl, 5,5-bis-(methoxycarbonyl)-pentyl, 6,6-bis-(methoxycarbonyl)-hexyl, 7,7-bis-(methoxycarbonyl)-heptyl, 8,8-bis-(methoxycarbonyl)-octyl, bis-(ethoxycarbonyl)-methyl, 2,2-bis-(ethoxycarbonyl)-ethyl, 3,3-bis-(ethoxycarbonyl)-propyl, 4,4-bis-(ethoxycarbonyl)-butyl, 5,5-bis-(ethoxycarbonyl)-hexyl.

An alkyl or alkenyl group that is monosubstituted by CN or CF₃ is preferably straight-chain. The substitution by CN or CF₃ can be in any desired position.

An alkyl or alkenyl group that is at least monosubstituted by halogen is preferably straight-chain. Halogen is preferably F or Cl, in case of multiple substitution preferably F. The resulting groups include also perfluorinated groups. In case of monosubstitution the F or Cl substituent can be in any desired position, but is preferably in ω-position. Examples for especially preferred straight-chain groups with a terminal F substituent are fluoromethyl, 2-fluoroethyl, 3-fluoropropyl, 4-fluorobutyl, 5-fluoropentyl, 6-fluorohexyl and 7-fluoroheptyl. Other positions of F are, however, not excluded.

R^{x} and R^{y} are preferably selected from H, straight-chain or branched alkyl with 1 to 12 C atoms.

—CY¹═CY²— is preferably —CH═CH—, —CF═CF— or —CH═C(CN)—.

Halogen is F, Cl, Br or I, preferably F or Cl.

R, R⁰, R¹, R² and R¹¹ can be an achiral or a chiral group. Particularly preferred chiral groups are 2-butyl (=1-methylpropyl), 2-methylbutyl, 2-methylpentyl, 3-methylpentyl, 2-ethylhexyl, 2-propylpentyl, in particular 2-methylbutyl, 2-methylbutoxy, 2-methylpentoxy, 3-methylpentoxy, 2-ethylhexoxy, 1-methylhexoxy, 2-octyloxy, 2-oxa-3-methylbutyl, 3-oxa-4-methylpentyl, 4-methylhexyl, 2-hexyl, 2-octyl, 2-nonyl, 2-decyl, 2-dodecyl, 6-methoxyoctoxy, 6-methyloctoxy, 6-methyloctanoyloxy, 5-methylheptyloxycarbonyl, 2-methylbutyryloxy, 3-methylvaleroyloxy, 4-methylhexanoyloxy, 2-chlorpropionyloxy, 2-chloro-3-methylbutyryloxy, 2-chloro-4-methylvaleryloxy, 2-chloro-3-methylvaleryloxy, 2-methyl-3-oxapentyl, 2-methyl-3-oxahexyl, 1-methoxypropyl-2-oxy, 1-ethoxypropyl-2-oxy, 1-propoxypropyl-2-oxy, 1-butoxypropyl-2-oxy, 2-fluorooctyloxy, 2-fluorodecyloxy, 1,1,1-trifluoro-2-octyloxy, 1,1,1-trifluoro-2-octyl, 2-fluoromethyloctyloxy for example. Very preferred are 2-hexyl, 2-octyl, 2-octyloxy, 1,1,1-trifluoro-2-hexyl, 1,1,1-trifluoro-2-octyl and 1,1,1-trifluoro-2-octyloxy.

Preferred achiral branched groups are isopropyl, isobutyl (=methylpropyl), isopentyl (=3-methylbutyl), isopropoxy, 2-methyl-propoxy and 3-methylbutoxy.

In formulae I, DRM, MRM and their preferred subformulae, the polymerisable groups P, P¹ and P² denote a group that is capable of participating in a polymerisation reaction, like radical or ionic chain polymerisation, polyaddition or polycondensation, or capable of being grafted, for example by condensation or addition, to a polymer backbone in a polymer analogous reaction. Especially preferred are polymerisable groups for chain polymerisation reactions, like radical, cationic or anionic polymerisation. Very preferred are polymerisable groups comprising a C—C double or triple bond, and polymerisable groups capable of polymerisation by a ring-opening reaction, like oxetanes or epoxides.

Suitable and preferred polymerisable groups P, P¹ and P² include, without limitation, CH₂═CW¹—COO—, CH₂═CW¹—CO—,

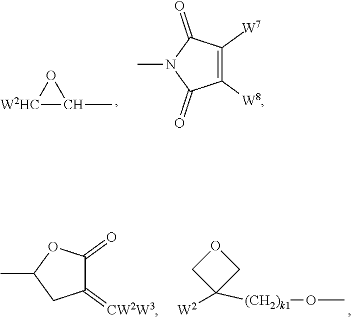

CH₂═CW²—(O)ₖ₁—, CH₃—CH═CH—O—, (CH₂═CH)₂CH—OCO—, (CH₂═CH—CH₂)₂CH—OCO—, (CH₂═CH)₂CH—O—, (CH₂═CH—CH₂)₂N—, (CH₂═CH—CH₂)₂N—CO—, HO—CW²W³—, HS—CW²W³—, HW²N—, HO—CW²W³—NH—, CH₂═CW¹—CO—NH—, CH₂═CH—(COO)ₖ₁-Phe-(O)ₖ₂—, CH₂═CH—(CO)ₖ₁-Phe-(O)ₖ₂—, Phe-CH═CH—, HOOC—, OCN—, and W⁴W⁵W⁶Si—, with W¹ being H, F, Cl, CN, CF₃, phenyl or alkyl with 1 to 5 C-atoms, in particular H, Cl or CH₃, W² and W³ being independently of each other H or alkyl with 1 to 5 C-atoms, in particular H, methyl, ethyl or n-propyl, W⁴, W⁵ and W⁶ being independently of each other Cl, oxaalkyl or oxacarbonylalkyl with 1 to 5 C-atoms, W⁷ and W⁸ being independently of each other H, Cl or alkyl with 1 to 5 C-atoms, Phe being 1,4-phenylene that is optionally substituted, preferably by one or more groups L as defined above (except for the meaning P-Sp-), and k₁ and k₂ being independently of each other 0 or 1.

Very preferred polymerisable groups P, P¹, and P² are selected from CH₂═CW¹—COO—, CH₂═CW¹—CO—,

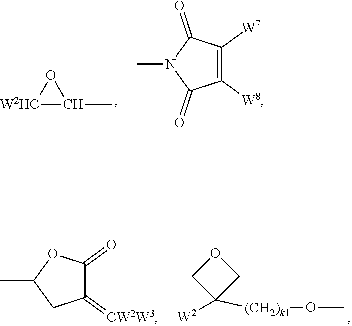

(CH₂═CH)₂CH—OCO—, (CH₂═CH—CH₂)₂CH—OCO—, (CH₂═CH)₂CH—O—, (CH₂═CH—CH₂)₂N—, (CH₂═CH—CH₂)₂N—CO—, HO—CW²W³—, HS—CW²W³—, HW²N—, HO—CW²W³—NH—, CH₂═CW¹—CO—NH—, CH₂═CH—(COO)ₖ₁-Phe-(O)ₖ₂—, CH₂═CH—(CO)ₖ₁-Phe-(O)ₖ₂—, Phe-CH═CH—, HOOC—, OCN—, and W⁴W⁵W⁶Si—, with W¹ being H, F, Cl, CN, CF₃, phenyl or alkyl with 1 to 5 C-atoms, in particular H, F, Cl or CH₃, W² and W³ being independently of each other H or alkyl with 1 to 5 C-atoms, in particular H, methyl, ethyl or n-propyl, W⁴, W⁵ and W⁶ being independently of each other Cl, oxaalkyl or oxacarbonylalkyl with 1 to 5 C-atoms, W⁷ and W⁸ being independently of each other H, Cl or alkyl with 1 to 5 C-atoms, Phe being 1,4-phenylene that is optionally substituted preferably by one or more groups L as defined above (except for the meaning P-Sp-), and k₁ and k₂ being independently of each other 0 or 1.

Most preferred polymerisable groups P, P¹ and P² are selected from CH₂═CH—COO—, CH₂═C(CH₃)—COO—, CH₂═CF—COO—, (CH₂═CH)₂CH—OCO—, (CH₂═CH)₂CH—O—,

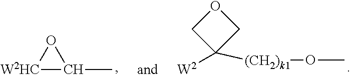

Further preferably P, P¹ and P² are selected from the group consisting of heptadiene, vinyloxy, acrylate, methacrylate, fluoroacrylate, chloroacrylate, oxetane and epoxide groups, and particularly preferably denote an acrylate, methacrylate or oxetane group.

Polymerisation can be carried out according to methods that are known to the ordinary expert and described in the literature, for example in D. J. Broer; G. Challa; G. N. Mol, Macromol. Chem, 1991, 192, 59.

In formulae I, DRM, MRM and their preferred subformulae, the spacer groups Sp, Sp¹ and Sp² are preferably selected of formula Sp′—X′, such that e.g. P-Sp- is P-Sp′—X′—, wherein
Sp′ is alkylene with 1 to 20 C atoms, preferably 1 to 12 C-atoms, which is optionally mono- or polysubstituted by F, Cl, Br, I or CN, and wherein one or more non-adjacent CH₂ groups are optionally replaced, in each case independently from one another, by —O—, —S—, —NH—, —NR⁰—, —SiR⁰⁰R⁰⁰⁰—, —CO—, —COO—, —OCO—, —OCO—O—, —S—CO—, —CO—S—, —NR⁰⁰—CO—O—, —O—CO—NR⁰⁰—, —NR⁰⁰—CO—NR⁰⁰—, —CH═CH— or —C≡C— in such a manner that O and/or S atoms are not linked directly to one another,
X′ is —O—, —S—, —CO—, —COO—, —OCO—, —O—COO—, —CO—NR^{x}—, —NR^{x}—CO—, —NR^{x}—CO—NR^{y}—, —OCH₂—, —CH₂O—, —SCH₂—, —CH₂S—, —CF₂O—, —OCF₂—, —CF₂S—, —SCF₂—, —CF₂CH₂—, —CH₂CF₂—, —CF₂CF₂—, —CH═N—, —N═CH—, —N═N—, —CH═CR^{x}—, —CY¹═CY², C≡C—, —CH═CH—COO—, —OCO—CH═CH— or a single bond,
R^{x} and R^{y} are independently of each other H or alkyl with 1 to 12 C-atoms, and
Y¹ and Y² are independently of each other H, F, Cl or CN.
X′ is preferably —O—, —S—CO—, —COO—, —OCO—, —O—COO—, —CO—NR⁰—, —NR⁰—CO—, —NR^{x}—CO—NR^{y}— or a single bond.

Typical groups Sp′ are, for example, —(CH₂)ₚ₁—, —(CH₂CH₂O)_{q1}—CH₂CH₂, —CH₂CH₂—S—CH₂CH₂— or —CH₂CH₂—NH—CH₂CH₂— or —(SiR^{x}R^{y}—O)ₚ₁—, with p1 being an integer from 2 to 12, q1 being an integer from 1 to 3 and R^{x} and R^{y} having the meanings given above.

Preferred groups Sp′ are ethylene, propylene, butylene, pentylene, hexylene, heptylene, octylene, nonylene, decylene, undecylene, dodecylene, octadecylene, ethyleneoxyethylene, methyleneoxy-butylene, ethylene-thioethylene, ethylene-N-methyl-iminoethylene, 1-methylalkylene, ethenylene, propenylene and butenylene for example.

Further preferred are compounds wherein the polymerisable group is directly attached to the mesogenic group without a spacer group Sp.

In case of compounds with multiple groups P-Sp-, P¹—Sp¹- etc., the multiple polymerisable groups P, P¹ and the multiple spacer groups Sp, Sp¹ can be identical or different from one another.

In another preferred embodiment the reactive compounds comprise one or more terminal groups R^{0,1,2} or substituents L or L¹⁻³ that are substituted by two or more polymerisable groups P or P-Sp- (multifunctional polymerisable groups). Suitable multifunctional polymerisable groups of this type are disclosed for example in U.S. Pat. No. 7,060,200 B1 or US 2006/0172090 A1. Very preferred are compounds comprising one or more multifunctional polymerisable groups selected from the following formulae:

—X-alkyl-CHP¹—CH₂—CH₂P² P1

—X′-alkyl-C(CH₂P¹)(CH₂P²)—CH₂P³ P2

—X′-alkyl-CHP¹CHP²—CH₂P³ P3

—X′-alkyl-C(CH₂P¹)(CH₂P²)—CₐₐH₂ₐₐ₊₁ P4

—X′-alkyl-CHP¹—CH₂P² P5

—X′-alkyl-CH P¹P² P6

—X′-alkyl-CP¹P²—CₐₐH₂ₐₐ₊₁ P7

—X′-alkyl-C(CH₂P¹)(CH₂P²)—CH₂H₂—C(CH₂P³)(CH₂P⁴)CH₂P⁵ P8

—X′-alkyl-CH((CH₂)ₐₐP¹)((CH₂)_{bb}P²) P9

—X′-alkyl-CH P¹CH P²—CₐₐH₂ₐₐ₊₁ P10

wherein
alkyl is straight-chain or branched alkylene having 1 to 12 C-atoms which is unsubstituted, mono- or polysubstituted by F, Cl, Br, I or CN, and wherein one or more non-adjacent CH₂ groups are optionally replaced, in each case independently from one another, by —O—, —S—, —NH—, —NR^{x}—, —SiR^{x}R^{y}—, —CO—, —COO—, —OCO—, —O—CO—O—, —S—CO—, —CO—S—, —SO₂—, —CO—NR^{x}—, —NR^{x}—CO—, —NR^{x}—CO—NR^{y}—, —CY¹═CY²— or —C≡C— in such a manner that O and/or S atoms are not linked directly to one another, with R^{x} and R^{y} having the meanings given above, or denotes a single bond,
aa and bb are independently of each other 0, 1, 2, 3, 4, 5 or 6,
X′ is as defined above, and
P¹⁻⁵ independently of each other have one of the meanings given for P above.

Preferably the RM mixture according to the present invention optionally comprises one or more chiral compounds. These chiral compounds may be non-mesogenic compounds or mesogenic compounds. Additionally, these chiral compounds, whether mesogenic or non-mesogenic, may be non-reactive, monoreactive or multireactive.

Preferably the utilized chiral compounds have each alone or in combination with each other an absolute value of the helical twisting power (|HTPₜₒₜₐₗ|) of 20 μm⁻¹ or more, preferably of 40 μm⁻¹ or more, more preferably in the range of 60 μm⁻¹ or more, most preferably in the range of 80 μm⁻¹ or more to 260 m⁻¹, in particular those disclosed in WO 98/00428.

Preferably, non-polymerisable chiral compounds are selected from the group of compounds of formulae C-I to C-III,

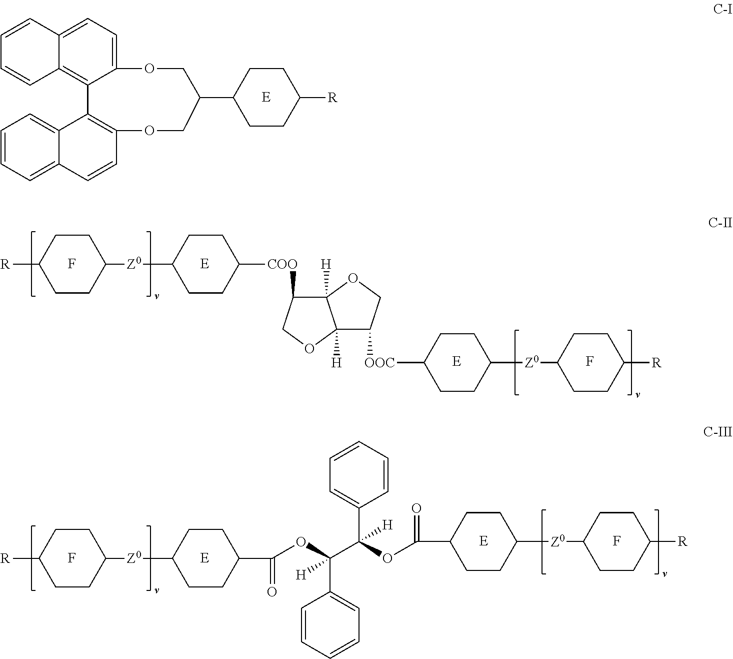

the latter ones including the respective (S,S) enantiomers,
wherein E and F are each independently 1,4-phenylene or trans-1,4-cyclo-hexylene, v is 0 or 1, Z⁰ is —COO—, —OCO—, —CH₂CH₂— or a single bond, and R is alkyl, alkoxy or alkanoyl with 1 to 12 C atoms.

Particularly preferred liquid-crystalline media comprise one or more chiral compounds, which do not necessarily have to show a liquid crystalline phase.

The compounds of formula C-II and their synthesis are described in WO 98/00428. Especially preferred is the compound CD-1, as shown in table D below. The compounds of formula C-III and their synthesis are described in GB 2 328 207.

Further, typically used chiral compounds are e.g. the commercially available R/S-5011, CD-1, R/S-811 and CB-15 (from Merck KGaA, Darmstadt, Germany).

The above mentioned chiral compounds R/S-5011 and CD-1 and the (other) compounds of formulae C-I, C-II and C-III exhibit a very high helical twisting power (HTP), and are therefore particularly useful for the purpose of the present invention.

The RM mixture preferably comprises 1 to 5, in particular 1 to 3, very preferably 1 or 2 chiral compounds, preferably selected from the above formula C-III, in particular CD-1, and/or formula C-III and/or R-5011 or S-5011, very preferably, the chiral compound is R-5011, S-5011 or CD-1.

Preferably the RM mixture optionally comprise one or more non-reactive chiral compound and/or one or more reactive chiral compounds, which are preferably selected from mono- and/or multireactive chiral compounds.

Suitable mesogenic reactive chiral compounds preferably comprise one or more ring elements, linked together by a direct bond or via a linking group and, where two of these ring elements optionally may be linked to each other, either directly or via a linking group, which may be identical to or different from the linking group mentioned. The ring elements are preferably selected from the group of four-, five-, six- or seven-, preferably of five- or six-, membered rings.

Suitable polymerisable chiral compounds and their synthesis are described in U.S. Pat. No. 7,223,450.

Preferred mono-reactive chiral compounds are selected from compounds of formula CRM.

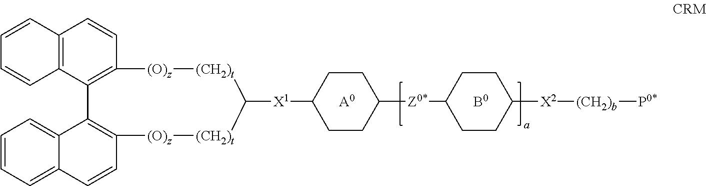

wherein
P^{0*} P, with P being a polymerisable group
A⁰ and B⁰ are, in case of multiple occurrence independently of one another, 1,4-phenylene that is unsubstituted or substituted with 1, 2, 3 or 4 groups L as defined above, or trans-1,4-cyclohexylene,
X¹ and X² are independently of each other —O—, —COO—, —OCO—, —O—CO—O— or a single bond,
Z^{0*} is, in case of multiple occurrence independently of one another, —COO—, —OCO—, —O—CO—O—, —OCH₂—, —CH₂O—, —CF₂O—, —OCF₂—, —CH₂CH₂—, —(CH₂)₄—, —CF₂CH₂—, —CH₂CF₂—, —CF₂CF₂—, —C≡C—, —CH═CH—, —CH═CH—COO—, —OCO—CH═CH— or a single bond,
t is, independently of each other 0, 1, 2 or 3,
a is 0, 1 or 2,
b is 0 or an integer from 1 to 12,
z is 0 or 1,
   and wherein the naphthalene rings can additionally be substituted with one or more identical or different groups L
   wherein
L is, independently of each other F, Cl, CN, halogenated alkyl, alkoxy, alkylcarbonyl, alkoxycarbonyl, alkylcarbonyloxy or alkoxycarbonyloxy with 1 to 5 C atoms.

The compounds of formula CRM are preferably selected from the group of compounds of formulae CRM-a.

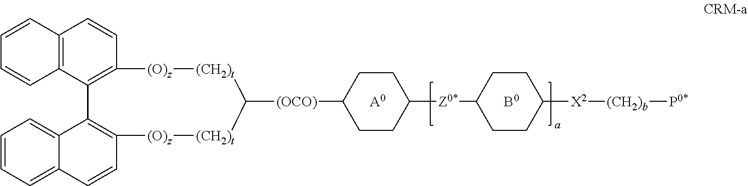

wherein A⁰, B⁰, Z^{0*}, P^{0*}, a and b have the meanings given in formula CRM or one of the preferred meanings given above and below, and (OCO) denotes —O—CO— or a single bond.

Especially preferred compounds of formula CRM are selected from the group consisting of the following subformulae:

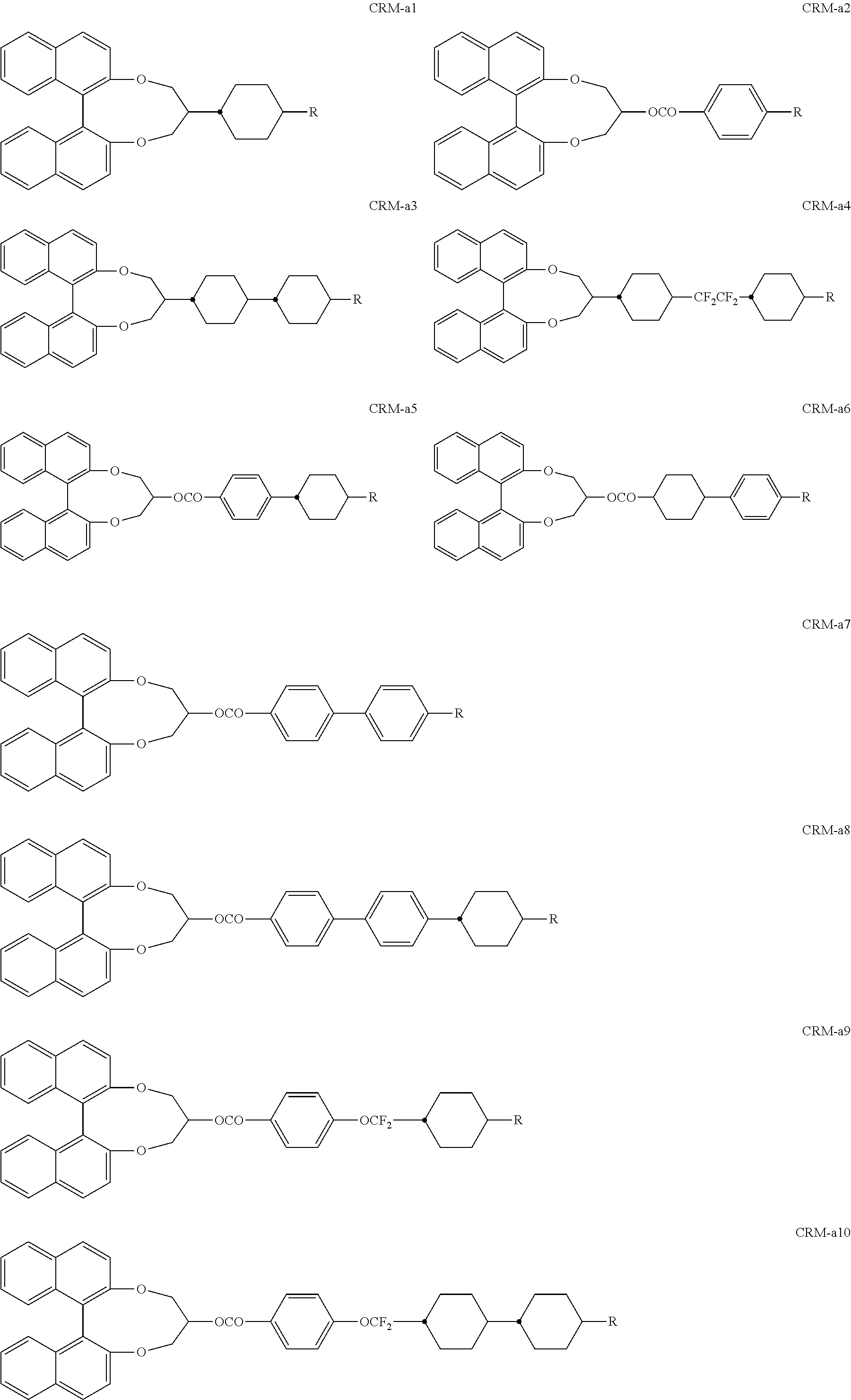

wherein R is —X²—(CH₂)ₓ—P^{0*} as defined in formula CRM-a, and the benzene and naphthalene rings are unsubstituted or substituted with 1, 2, 3 or 4 groups L as defined above and below.

The amount of chiral compounds in the liquid-crystalline medium is preferably from 1 to 20%, more preferably from 1 to 15%, even more preferably 1 to 10%, and most preferably 2 to 6%, by weight of the total mixture.

In a preferred embodiment the RM formulation comprises additionally one or more liquid crystalline monothiol compounds. Typical thiols used according to the present invention are of the following structure:

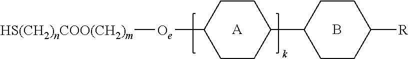

wherein
n denotes 1 to 6
m denotes 0 to 10
e denotes 0 or 1
k denotes 0 or 1

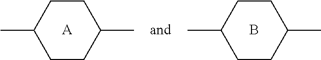

each independently

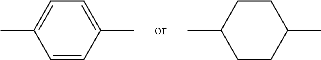

or another
6-membered 1-4 disubstituted ring which can also bear one or more lateral
groups like R or F, and
R denotes alkyl, alkenyl, oxyalkyl or oxyalkenyl.

Another object of the invention is an RM formulation comprising one or more compounds of formula I, or comprising an RM mixture, as described above and below, and further comprising one or more solvents and/or additives.

In a preferred embodiment the RM formulation comprises optionally one or more additives selected from the group consisting of polymerisation initiators, surfactants, stabilisers, catalysts, sensitizers, inhibitors, chain-transfer agents, co-reacting monomers, reactive thinners, surface-active compounds, lubricating agents, wetting agents, dispersing agents, hydrophobing agents, adhesive agents, flow improvers, degassing or defoaming agents, deaerators, diluents, reactive diluents, auxiliaries, colourants, dyes, pigments and nanoparticles.

In another preferred embodiment the RM formulation optionally comprises one or more additives selected from polymerisable non-mesogenic compounds (reactive thinners). The amount of these additives in the RM formulation is preferably from 0 to 30%, very preferably from 0 to 25%.

The reactive thinners used are not only substances which are referred to in the actual sense as reactive thinners, but also auxiliary compounds already mentioned above which contain one or more complementary reactive units, for example hydroxyl, thiol-, or amino groups, via which a reaction with the polymerizable units of the liquid-crystalline compounds can take place.

The substances which are usually capable of photopolymerization include, for example, mono-, bi- and polyfunctional compounds containing at least one olefinic double bond. Examples thereof are vinyl esters of carboxylic acids, for example of lauric, myristic, palmitic and stearic acid, and of dicarboxylic acids, for example of succinic acid, adipic acid, allyl and vinyl ethers and methacrylic and acrylic esters of monofunctional alcohols, for example of lauryl, myristyl, palmityl and stearyl alcohol, and diallyl and divinyl ethers of bifunctional alcohols, for example ethylene glycol and 1,4-butanediol.

Also suitable are, for example, methacrylic and acrylic esters of polyfunctional alcohols, in particular those which contain no further functional groups, or at most ether groups, besides the hydroxyl groups. Examples of such alcohols are bifunctional alcohols, such as ethylene glycol, propylene glycol and their more highly condensed representatives, for example diethylene glycol, triethylene glycol, dipropylene glycol, tripropylene glycol etc., butanediol, pentanediol, hexanediol, neopentyl glycol, alkoxylated phenolic compounds, such as ethoxylated and propoxylated bisphenols, cyclohexanedimethanol, trifunctional and polyfunctional alcohols, such as glycerol, trimethylolpropane, butanetriol, trimethylolethane, pentaerythritol, ditrimethylolpropane, dipentaerythritol, sorbitol, mannitol, and the corresponding alkoxylated, in particular ethoxylated and propoxylated alcohols.

Other suitable reactive thinners are polyester (meth)acrylates, which are the (meth)acrylic ester of polyesterols.

Examples of suitable polyesterols are those which can be prepared by esterification of polycarboxylic acids, preferably dicarboxylic acids, using polyols, preferably diols. The starting materials for such hydroxyl-containing polyesters are known to the person skilled in the art. Dicarboxylic acids which can be employed are succinic, glutaric acid, adipic acid, sebacic acid, o-phthalic acid and isomers and hydrogenation products thereof, and esterifiable and transesterifiable derivatives of said acids, for example anhydrides and dialkyl esters. Suitable polyols are the abovementioned alcohols, preferably ethyleneglycol, 1,2- and 1,3-propylene glycol, 1,4-butanediol, 1,6-hexanediol, neopentyl glycol, cyclohexanedimethanol and polyglycols of the ethylene glycol and propylene glycol type.

Suitable reactive thinners are furthermore 1,4-divinylbenzene, triallyl cyanurate, acrylic esters of tricyclodecenyl alcohol of the following formula

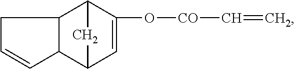 [SMILES: C=CC(=O)OC1=CC2CC1C1CC=CC21]

also known under the name dihydrodicyclopentadienyl acrylate, and the allyl esters of acrylic acid, methacrylic acid and cyanoacrylic acid.

Of the reactive thinners which are mentioned by way of example, those containing photopolymerizable groups are used in particular and in view of the abovementioned preferred compositions.

This group includes, for example, dihydric and polyhydric alcohols, for example ethylene glycol, propylene glycol and more highly condensed representatives thereof, for example diethylene glycol, triethylene glycol, dipropylene glycol, tripropylene glycol etc., butanediol, pentanediol, hexanediol, neopentyl glycol, cyclohexanedimethanol, glycerol, trimethylolpropane, butanetriol, trimethylolethane, pentaerythritol, ditrimethylolpropane, dipentaerythritol, sorbitol, mannitol and the corresponding alkoxylated, in particular ethoxylated and propoxylated alcohols.

The group furthermore also includes, for example, alkoxylated phenolic compounds, for example ethoxylated and propoxylated bisphenols.

These reactive thinners may furthermore be, for example, epoxide or urethane (meth)acrylates.

Epoxide (meth)acrylates are, for example, those as obtainable by the reaction, known to the person skilled in the art, of epoxidized olefins or poly- or diglycidyl ether, such as bisphenol A diglycidyl ether, with (meth)acrylic acid.

Urethane (meth)acrylates are, in particular, the products of a reaction, likewise known to the person skilled in the art, of hydroxylalkyl (meth)acrylates with poly- or diisocyanates.

Such epoxide and urethane (meth)acrylates are included amongst the compounds listed above as “mixed forms”.

If reactive thinners are used, their amount and properties must be matched to the respective conditions in such a way that, on the one hand, a satisfactory desired effect, for example the desired colour of the composition according to the invention, is achieved, but, on the other hand, the phase behaviour of the liquid-crystalline composition is not excessively impaired. The low-crosslinking (high-crosslinking) liquid-crystalline compositions can be prepared, for example, using corresponding reactive thinners which have a relatively low (high) number of reactive units per molecule.

The group of diluents include, for example:

C1-C4-alcohols, for example methanol, ethanol, n-propanol, isopropanol, butanol, isobutanol, sec-butanol and, in particular, the C5-C12-alcohols n-pentanol, n-hexanol, n-heptanol, n-octanol, n-nonanol, n-decanol, n-undecanol and n-dodecanol, and isomers thereof, glycols, for example 1,2-ethylene glycol, 1,2- and 1,3-propylene glycol, 1,2-, 2,3- and 1,4-butylene glycol, di- and triethylene glycol and di- and tripropylene glycol, ethers, for example methyl tert-butyl ether, 1,2-ethylene glycol mono- and dimethyl ether, 1,2-ethylene glycol mono- and -diethylether, 3-methoxypropanol, 3-isopropoxypropanol, tetrahydrofuran and dioxane, ketones, for example acetone, methyl ethyl ketone, methyl isobutyl ketone and diacetone alcohol (4-hydroxy-4-methyl-2-pentanone), C1-C5-alkyl esters, for example methyl acetate, ethyl acetate, propyl acetate, butyl acetate and amyl acetate, aliphatic and aromatic hydrocarbons, for example pentane, hexane, heptane, octane, isooctane, petroleum ether, toluene, xylene, ethylbenzene, tetralin, decalin, dimethylnaphthalene, white spirit, Shellsol® and Solvesso® mineral oils, for example gasoline, kerosine, diesel oil and heating oil, but also natural oils, for example olive oil, soya oil, rapeseed oil, linseed oil and sunflower oil.

It is of course also possible to use mixtures of these diluents in the compositions according to the invention.

So long as there is at least partial miscibility, these diluents can also be mixed with water. Examples of suitable diluents here are C1-C4-alcohols, for example methanol, ethanol, n-propanol, isopropanol, butanol, isobutanol and sec-butanol, glycols, for example 1,2-ethylene glycol, 1,2- and 1,3-propylene glycol, 1,2-, 2,3- and 1,4-butylene glycol, di- and triethylene glycol, and di- and tripropylene glycol, ethers, for example tetrahydrofuran and dioxane, ketones, for example acetone, methyl ethyl ketone and diacetone alcohol (4-hydroxy-4-methyl-2-pentanone), and C1-C4-alkyl esters, for example methyl, ethyl, propyl and butyl acetate.

The diluents are optionally employed in a proportion of from about 0 to 10.0% by weight, preferably from about 0 to 5.0% by weight, based on the total weight of the RM formulation.

The antifoams and deaerators (c1)), lubricants and flow auxiliaries (c2)), thermally curing or radiation-curing auxiliaries (c3)), substrate wetting auxiliaries (c4)), wetting and dispersion auxiliaries (c5)), hydrophobicizing agents (c6)), adhesion promoters (c7)) and auxiliaries for promoting scratch resistance (c8)) cannot strictly be delimited from one another in their action.

For example, lubricants and flow auxiliaries often also act as antifoams and/or deaerators and/or as auxiliaries for improving scratch resistance. Radiation-curing auxiliaries can also act as lubricants and flow auxiliaries and/or deaerators and/or as substrate wetting auxiliaries. In individual cases, some of these auxiliaries can also fulfil the function of an adhesion promoter (c8)).

Corresponding to the above-said, a certain additive can therefore be classified in a number of the groups c1) to c8) described below.

The antifoams in group c1) include silicon-free and silicon-containing polymers. The silicon-containing polymers are, for example, unmodified or modified polydialkylsiloxanes or branched copolymers, comb or block copolymers comprising polydialkylsiloxane and polyether units, the latter being obtainable from ethylene oxide or propylene oxide.

The deaerators in group c1) include, for example, organic polymers, for example polyethers and polyacrylates, dialkylpolysiloxanes, in particular dimethylpolysiloxanes, organically modified polysiloxanes, for example arylalkyl-modified polysiloxanes, and fluorosilicones.

The action of the antifoams is essentially based on preventing foam formation or destroying foam that has already formed. Antifoams essentially work by promoting coalescence of finely divided gas or air bubbles to give larger bubbles in the medium to be deaerated, for example the compositions according to the invention, and thus accelerate escape of the gas (of the air). Since antifoams can frequently also be employed as deaerators and vice versa, these additives have been included together under group c1).

Such auxiliaries are, for example, commercially available from Tego as TEGO® Foamex 800, TEGO® Foamex 805, TEGO® Foamex 810, TEGO® Foamex 815, TEGO® Foamex 825, TEGO® Foamex 835, TEGO® Foamex 840, TEGO® Foamex 842, TEGO® Foamex 1435, TEGO® Foamex 1488, TEGO® Foamex 1495, TEGO® Foamex 3062, TEGO® Foamex 7447, TEGO® Foamex 8020, Tego® Foamex N, TEGO® Foamex K 3, TEGO® Antifoam 2-18, TEGO® Antifoam 2-18, TEGO® Antifoam 2-57, TEGO® Antifoam 2-80, TEGO® Antifoam 2-82, TEGO® Antifoam 2-89, TEGO® Antifoam 2-92, TEGO® Antifoam 14, TEGO® Antifoam 28, TEGO® Antifoam 81, TEGO® Antifoam D 90, TEGO® Antifoam 93, TEGO® Antifoam 200, TEGO® Antifoam 201, TEGO® Antifoam 202, TEGO® Antifoam 793, TEGO® Antifoam 1488, TEGO® Antifoam 3062, TEGOPREN® 5803, TEGOPREN® 5852, TEGOPREN® 5863, TEGOPREN® 7008, TEGO® Antifoam 1-60, TEGO® Antifoam 1-62, TEGO® Antifoam 1-85, TEGO® Antifoam 2-67, TEGO® Antifoam WM 20, TEGO® Antifoam 50, TEGO® Antifoam 105, TEGO® Antifoam 730, TEGO® Antifoam MR 1015, TEGO® Antifoam MR 1016, TEGO® Antifoam 1435, TEGO® Antifoam N, TEGO® Antifoam KS 6, TEGO® Antifoam KS 10, TEGO® Antifoam KS 53, TEGO® Antifoam KS 95, TEGO® Antifoam KS 100, TEGO® Antifoam KE 600, TEGO® Antifoam KS 911, TEGO® Antifoam MR 1000, TEGO® Antifoam KS 1100, Tego® Airex 900, Tego® Airex 910, Tego® Airex 931, Tego® Airex 935, Tego® Airex 936, Tego® Airex 960, Tego® Airex 970, Tego® Airex 980 and Tego® Airex 985 and from BYK as BYK®-011, BYK®-019, BYK®-020, BYK®-021, BYK®-022, BYK®-023, BYK®-024, BYK®-025, BYK®-027, BYK®-031, BYK®-032, BYK®-033, BYK®-034, BYK®-035, BYK®-036, BYK®-037, BYK®-045, BYK®-051, BYK®-052, BYK®-053, BYK®-055, BYK®-057, BYK®-065, BYK®-066, BYK®-070, BYK®-080, BYK®-088, BYK®-141 and BYK®-A 530.

The auxiliaries in group c1) are optionally employed in a proportion of from about 0 to 3.0% by weight, preferably from about 0 to 2.0% by weight, based on the total weight of the RM formulation.

In group c2), the lubricants and flow auxiliaries typically include silicon-free, but also silicon-containing polymers, for example polyacrylates or modifiers, low-molecular-weight polydialkylsiloxanes. The modification consists in some of the alkyl groups having been replaced by a wide variety of organic radicals. These organic radicals are, for example, polyethers, polyesters or even long-chain alkyl radicals, the former being used the most frequently.

The polyether radicals in the correspondingly modified polysiloxanes are usually built up from ethylene oxide and/or propylene oxide units. Generally, the higher the proportion of these alkylene oxide units in the modified polysiloxane, the more hydrophilic is the resultant product.

Such auxiliaries are, for example, commercially available from Tego as TEGO® Glide 100, TEGO® Glide ZG 400, TEGO® Glide 406, TEGO® Glide 410, TEGO® Glide 411, TEGO® Glide 415, TEGO® Glide 420, TEGO® Glide 435, TEGO® Glide 440, TEGO® Glide 450, TEGO® Glide A 115, TEGO® Glide B 1484 (can also be used as antifoam and deaerator), TEGO® Flow ATF, TEGO® Flow 300, TEGO® Flow 460, TEGO® Flow 425 and TEGO® Flow ZFS 460. Suitable radiation-curable lubricants and flow auxiliaries, which can also be used to improve the scratch resistance, are the products TEGO® Rad 2100, TEGO® Rad 2200, TEGO® Rad 2500, TEGO® Rad 2600 and TEGO® Rad 2700, which are likewise obtainable from TEGO.

Such-auxiliaries are available, for example, from BYK as BYK®-300 BYK®-306, BYK®-307, BYK®-310, BYK®-320, BYK®-333, BYK®-341, Byk® 354, Byk®361, Byk®361 N, BYK®388.

The auxiliaries in group c2) are optionally employed in a proportion of from about 0 to 3.0% by weight, preferably from about 0 to 2.0% by weight, based on the total weight of the RM formulation.

In group c3), the radiation-curing auxiliaries include, in particular, polysiloxanes having terminal double bonds which are, for example, a constituent of an acrylate group. Such auxiliaries can be crosslinked by actinic or, for example, electron radiation. These auxiliaries generally combine a number of properties together. In the uncrosslinked state, they can act as antifoams, deaerators, lubricants and flow auxiliaries and/or substrate wetting auxiliaries, while, in the crosslinked state, they increase, in particular, the scratch resistance, for example of coatings or films which can be produced using the compositions according to the invention. The improvement in the gloss properties, for example of precisely those coatings or films, is regarded essentially as a consequence of the action of these auxiliaries as antifoams, deaerators and/or lubricants and flow auxiliaries (in the uncrosslinked state).

Examples of suitable radiation-curing auxiliaries are the products TEGO® Rad 2100, TEGO® Rad 2200, TEGO® Rad 2500, TEGO® Rad 2600 and TEGO® Rad 2700 available from TEGO and the product BYK®-371 available from BYK.

Thermally curing auxiliaries in group c3) contain, for example, primary OH groups which are able to react with isocyanate groups, for example of the binder.

Examples of thermally curing auxiliaries which can be used are the products BYK®-370, BYK®-373 and BYK®-375 available from BYK.

The auxiliaries in group c3) are optionally employed in a proportion of from about 0 to 5.0% by weight, preferably from about 0 to 3.0% by weight, based on the total weight of the RM formulation.

The substrate wetting auxiliaries in group c4) serve, in particular, to increase the wettability of the substrate to be printed or coated, for example, by printing inks or coating compositions, for example compositions according to the invention. The generally attendant improvement in the lubricant and flow behaviour of such printing inks or coating compositions has an effect on the appearance of the finished (for example crosslinked) print or coating.

A wide variety of such auxiliaries are commercially available, for example from Tego as TEGO® Wet KL 245, TEGO® Wet 250, TEGO® Wet 260 and TEGO® Wet ZFS 453 and from BYK as BYK®-306, BYK®-307, BYK®-310, BYK®-333, BYK®-344, BYK®-345, BYK®-346 and Byk®-348.

The auxiliaries in group c4) are optionally employed in a proportion of from about 0 to 3.0% by weight, preferably from about 0 to 1.5% by weight, based on the total weight of the liquid-crystalline composition.

The wetting and dispersion auxiliaries in group c5) serve, in particular, to prevent the flooding and floating and the sedimentation of pigments and are therefore, if necessary, suitable in particular in pigmented compositions according to the invention.

These auxiliaries stabilize pigment dispersions essentially through electrostatic repulsion and/or steric hindrance of the pigment particles containing these additives, where, in the latter case, the interaction of the auxiliary with the ambient medium (for example binder) plays a major role.

Since the use of such wetting and dispersion auxiliaries is common practice, for example in the technical area of printing inks and paints, the selection of a suitable auxiliary of this type generally does not present the person skilled in the art with any difficulties, if they are used.

Such wetting and dispersion auxiliaries are commercially available, for example from Tego, as TEGO® Dispers 610, TEGO® Dispers 610 S, TEGO® Dispers 630, TEGO® Dispers 700, TEGO® Dispers 705, TEGO® Dispers 710, TEGO® Dispers 720 W, TEGO® Dispers 725 W, TEGO® Dispers 730 W, TEGO® Dispers 735 W and TEGO® Dispers 740 W and from BYK as Disperbyk®, Disperbyk®-107, Disperbyk®-108, Disperbyk®-110, Disperbyk®-111, Disperbyk®-115, Disperbyk®-130, Disperbyk®-160, Disperbyk®-161, Disperbyk®-162, Disperbyk®-163, Disperbyk®-164, Disperbyk®-165, Disperbyk®-166, Disperbyk®-167, Disperbyk®-170, Disperbyk®-174, Disperbyk®-180, Disperbyk®-181, Disperbyk®-182, Disperbyk®-183, Disperbyk®-184, Disperbyk®-185, Disperbyk®-190, Anti-Terra®-U, Anti-Terra®-U 80, Anti-Terra®-P, Anti-Terra®-203, Anti-Terra®-204, Anti-Terra®-206, BYK®-151, BYK®-154, BYK®-155, BYK®-P 104 S, BYK®-P 105, Lactimon®, Lactimon®-WS and Bykumen®.

The amount of the auxiliaries in group c5) used on the mean molecular weight of the auxiliary. In any case, a preliminary experiment is therefore advisable, but this can be accomplished simply by the person skilled in the art.

The hydrophobicizing agents in group c6) can be used to give water-repellent properties to prints or coatings produced, for example, using compositions according to the invention. This prevents or at least greatly suppresses swelling due to water absorption and thus a change in, for example, the optical properties of such prints or coatings. In addition, when the composition is used, for example, as a printing ink in offset printing, water absorption can thereby be prevented or at least greatly reduced.

Such hydrophobicizing agents are commercially available, for example, from Tego as Tego® Phobe WF, Tego® Phobe 1000, Tego® Phobe 1000 S, Tego® Phobe 1010, Tego® Phobe 1030, Tego® Phobe 1010, Tego® Phobe 1010, Tego® Phobe 1030, Tego® Phobe 1040, Tego® Phobe 1050, Tego® Phobe 1200, Tego® Phobe 1300, Tego® Phobe 1310 and Tego® Phobe 1400.

The auxiliaries in group c6) are optionally employed in a proportion of from about 0 to 5.0% by weight, preferably from about 0 to 3.0% by weight, based on the total weight of the RM formulation.

Adhesion promoters from group c7) serve to improve the adhesion of two interfaces in contact. It is directly evident from this that essentially the only fraction of the adhesion promoter that is effective is that located at one or the other or at both interfaces. If, for example, it is desired to apply liquid or pasty printing inks, coating compositions or paints to a solid substrate, this generally means that the adhesion promoter must be added directly to the latter or the substrate must be pre-treated with the adhesion promoters (also known as priming), i.e. this substrate is given modified chemical and/or physical surface properties.

If the substrate has previously been primed with a primer, this means that the interfaces in contact are that of the primer on the one hand and of the printing ink or coating composition or paint on the other hand. In this case, not only the adhesion properties between the substrate and the primer, but also between the substrate and the printing ink or coating composition or paint play a part in adhesion of the overall multilayer structure on the substrate.

Adhesion promoters in the broader sense which may be mentioned are also the substrate wetting auxiliaries already listed under group c4), but these generally do not have the same adhesion promotion capacity.

In view of the widely varying physical and chemical natures of substrates and of printing inks, coating compositions and paints intended, for example, for their printing or coating, the multiplicity of adhesion promoter systems is not surprising.

Adhesion promoters based on silanes are, for example, 3-aminopropyltrimethoxysilane, 3-aminopropyltriethoxysilane, 3-aminopropylmethyldiethoxysilane, N-aminoethyl-3-aminopropyltrimethoxysilane, N-aminoethyl-3-aminopropylmethyldimethoxysilane, N-methyl-3-aminopropyltrimethoxysilane, 3-ureidopropyltriethoxysilane, 3-methacryloyloxypropyltrimethoxysilane, 3-glycidyloxypropyltrimethoxysilane, 3-mercaptopropyltrimethoxysilane, 3-chloropropyltrimethoxysilane and vinyltrimethoxysilane. These and other silanes are commercially available from Hils, for example under the tradename DYNASILAN®.

Corresponding technical information from the manufacturers of such additives should generally be used or the person skilled in the art can obtain this information in a simple manner through corresponding preliminary experiments.

However, if these additives are to be added as auxiliaries from group c7) to the RM formulations according to the invention, their proportion optionally corresponds to from about 0 to 5.0% by weight, based on the total weight of the RM formulation. These concentration data serve merely as guidance, since the amount and identity of the additive are determined in each individual case by the nature of the substrate and of the printing/coating composition. Corresponding technical information is usually available from the manufacturers of such additives for this case or can be determined in a simple manner by the person skilled in the art through corresponding preliminary experiments.

The auxiliaries for improving the scratch resistance in group c8) include, for example, the abovementioned products TEGO® Rad 2100, TEGO® Rad 2200, TEGO® Rad 2500, TEGO® Rad 2600 and TEGO® Rad 2700, which are available from Tego.

For these auxiliaries, the amount data given for group c3) are likewise suitable, i.e. these additives are optionally employed in a proportion of from about 0 to 5.0% by weight, preferably from about 0 to 3.0% by weight, based on the total weight of the liquid-crystalline composition.

Examples which may be mentioned of light, heat and/or oxidation stabilizers are the following:

alkylated monophenols, such as 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which have a linear or branched side chain, for example 2,6-dinonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol and mixtures of these compounds, alkylthiomethylphenols, such as 2,4-dioctylthiomethyl-6-tert-butylphenol, 2,4-dioctylthiomethyl-6-methylphenol, 2,4-dioctylthiomethyl-6-ethylphenol and 2,6-didodecylthiomethyl-4-nonylphenol, Hydroquinones and alkylated hydroquinones, such as 2,6-di-tert-butyl-4-methoxyphenol, 2,5-di-tert-butylhydroquinone, 2,5-di-tert-amylhydrocrainone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate and bis(3,5-di-tert-butyl-4-hydroxyphenyl)adipate,

Tocopherols, such as α-tocopherol, β-tocopherol, γ-tocopherol, δ-tocopherol and mixtures of these compounds, and tocopherol derivatives, such as tocopheryl acetate, succinate, nicotinate and polyoxyethylenesuccinate (“tocofersolate”), hydroxylated diphenyl thioethers, such as 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol) and 4,4′-bis(2,6-dimethyl-4-hydroxyphenyl)disulfide, Alkylidenebisphenols, such as 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methylphenyl)-3-n-dodecyl-mercaptobutane, ethylene glycol bis[3,3-bis(3′-tert-butyl-4′-hydroxyphenyl)butyrate], bis(3-tert-butyl-4-hydroxy-5-methylphenyl)dicyclopentadiene, bis[2-(3′-tert-butyl-2′-hydroxy-5′-methylbenzyl)-6-tert-butyl-4-methylphenyl]terephthalate, 1,1-bis(3,5-dimethyl-2-hydroxyphenyl)butane, 2,2-bis(3,5-di-tert-butyl-4-hydroxyphenyl)propane, 2,2-bis(5-tert-butyl-4-hydroxy-2-methylphenyl)-4-n-dodecyl-mercaptobutane and 1,1,5,5-tetrakis(5-tert-butyl-4-hydroxy-2-methylphenyl)pentane,

O—, N- and S-benzyl compounds, such as 3,5,3′,5′-tetra-tert-butyl-4,4′-dihydroxydibenzyl ether, octadecyl 4-hydroxy-3,5-dimethylbenzylmercaptoacetate, tridecyl 4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide and isooctyl-3,5-di-tert-butyl-4-hydroxybenzylmercaptoacetate,

aromatic hydroxybenzyl compounds, such as 1,3,5-tris(3,5-di-tert-butyl-4-hydroxybenzyl)-2,4,6-trimethyl-benzene, 1,4-bis(3,5-di-tert-butyl-4-hydroxybenzyl)-2,3,5,6-tetramethyl-benzene and 2,4,6-tris(3,5-di-tert-butyl-4-hydroxybenzyl)phenol,

Triazine compounds, such as 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxyphenylethyl)-1,3,5-triazine, 1,3,5-tris-(3,5-di-tert-butyl-4-hydroxyphenylpropionyl)hexahydro-1,3,5-triazine, 1,3,5-tris(3,5-dicyclohexyl-4-hydroxybenzyl)isocyanurate and 1,3,5-tris(2-hydroxyethyl)isocyanurate,

Benzylphosphonates, such as dimethyl 2,5-di-tert-butyl-4-hydroxybenzylphosphonate, diethyl 3,5-di-tert-butyl-4-hydroxybenzylphosphonate, dioctadecyl 3,5-di-tert-butyl-4-hydroxybenzylphosphonate and dioctadecyl 5-tert-butyl-4-hydroxy-3-methylbenzylphosphonate,

Acylaminophenols, such as 4-hydroxylauroylanilide, 4-hydroxystearoylanilide and octyl N-(3,5-di-tert-butyl-4-hydroxyphenyl)carbamate,

Propionic and acetic esters, for example of monohydric or polyhydric alcohols, such as methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxalamide, 3-thiaundecanol, 3-thiapentadecanol, trimethylhexanediol, trimethylolpropane and 4-hydroxymethyl-1-phospha-2,6,7-trioxabicyclo[2.2.2]-octane, Propionamides based on amine derivatives, such as N,N′-bis(3,5-di-tert-butyl-4-hydroxyphenylpropionyl)hexamethylenediamine, N,N′-bis(3,5-di-tert-butyl-4-hydroxyphenylpropionyl)trimethylenediamine and N,N′-bis(3,5-di-tert-butyl-4-hydroxyphenylpropionyl)hydrazine,

Ascorbic acid (Vitamin C) and ascorbic acid derivatives, such as ascorbyl palmitate, laurate and stearate, and ascorbyl sulfate and phosphate,

Antioxidants based on amine compounds, such as N,N′-diisopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine, 4-(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octyl-substituted diphenylamine, such as p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis[4-methoxyphenyl)amine, 2,6-di-tert-butyl-4-dimethylaminomethylphenol, 2,4-diaminodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octyl-substituted N-phenyl-1-naphthylamine, a mixture of mono- and dialkylated tert-butyl/tert-octyldiphenylamine, a mixture of mono- and dialkylated nonyldiphenylamine, a mixture of mono- and dialkylated dodecyldiphenylamine, a mixture of mono- and dialkylated isopropyl/isohexyldiphenylamine, a mixture of mono- and dialkylated tert-butyldiphenylamine, 2,3-dihydro-3,3-dimethyl-4H-1,4-benzothiazine, phenothiazine, a mixture of mono- and dialkylated tert-butyl/tert-octylphenothiazine, a mixture of mono- and dialkylated tert-octylphenothiazine, N-allylphenothiazine, N,N,N′,N′-tetraphenyl-1,4-diaminobut-2-ene, N,N-bis(2,2,6,6-tetramethylpiperidin-4-yl)hexamethylenediamine, bis(2,2,6,6-tetramethylpiperidin-4-yl)sebacate, 2,2,6,6-tetramethylpiperidin-4-one and 2,2,6,6-tetramethylpiperidin-4-ol,

Phosphines, Phosphites and phosphonites, such as triphenylphosnine triphenylphosphite, diphenyl alkyl phosphite, phenyl dialkyl phosphite, tris(nonylphenyl)phosphite, trilauryl phosphite, trioctadecyl phosphite, distearyl pentaerythritol diphosphite, tris(2,4-di-tert-butylphenyl)phosphite, diisodecyl pentaerythritol diphosphite, bis(2,4-di-tert-butylphenyl)pentaerythritol diphosphite, bis(2,6-di-tert-butyl-4-methylphenyl)pentaerythritol diphosphite, diisodecyloxy pentaerythritol diphosphite, bis(2,4-di-tert-butyl-6-methylphenyl)pentaerythritol diphosphite, bis(2,4,6-tris(tert-butylphenyl))pentaerythritol diphosphite, tristearyl sorbitol triphosphite, tetrakis(2,4-di-tert-butylphenyl)4,4′-biphenylenediphosphonite, 6-isooctyloxy-2,4,8,10-tetra-tert-butyl-12H-dibenz[d,g]-1,3,2-dioxaphosphocine, 6-fluoro-2,4,8,10-tetra-tert-butyl-12-methyl-dibenz[d,g]-1,3,2-dioxaphosphocine, bis(2,4-di-tert-butyl-6-methylphenyl)methyl phosphite and bis(2,4-di-tert-butyl-6-methylphenyl)ethyl phosphite,

2-(2′-Hydroxyphenyl)benzotriazoles, such as 2-(2′-hydroxy-5′-methylphenyl)benzotriazole, 2-(3′,5′-di-tert-butyl-2′-hydroxyphenyl)benzotriazole, 2-(5′-tert-butyl-2′-hydroxyphenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chlorobenzotriazole, 2-(3′-tert-butyl-2′-hydroxy-5′-methylphenyl)-5-chlorobenzotriazole, 2-(3′-sec-butyl-5′-tert-butyl-2′-hydroxyphenyl)benzotriazole, 2-(2′-hydroxy-4′-octyloxyphenyl)benzotriazole, 2-(3′,5′-di-tert-amyl-2′-hydroxyphenyl)benzotriazole, 2-(3,5′-bis-(α,α-dimethylbenzyl)-2′-hydroxyphenyl)benzotriazole, a mixture of 2-(3′-tert-butyl-2′-hydroxy-5′-(2-octyloxycarbonylethyl)phenyl)-5-chlorobenzotriazole, 2-(3′-tert-butyl-5′-[2-(2-ethylhexyloxy)carbonylethyl]-2′-hydroxy phenyl)-5-chlorobenzotriazole, 2-(3′-tert-butyl-2′-hydroxy-5′-(2-methoxycarbonylethyl)phenyl)-5-chlorobenzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxy phenyl)benzotriazole, 2-(3′-dodecyl-2′-hydroxy-5′-methylphenyl)benzotriazole and 2-(3′-tert-butyl-2′-hydroxy-5′-(2-isooctyloxycarbonylethyl)phenyl benzotriazole, 2,2′-methylenebis[4-(1,1,3,3-tetramethylbutyl)-6-benzotriazol-2-ylphenol]; the product of complete esterification of 2-[3′-tert-butyl-5′-(2-methoxycarbonylethyl)-2′-hydroxyphenyl]-2H-benzotriazole with polyethylene glycol 300; [R-CH2CH2-COO(CH2)3□2, where R=3′-tert-butyl-4′-hydroxy-5′-2H-benzotriazol-2-ylphenyl], sulfur-containing peroxide scavengers and sulfur-containing antioxidants, such as esters of 3,3′-thiodipropionic acid, for example the lauryl, stearyl, myristyl and tridecyl esters, mercaptobenzimidazole and the zinc salt of 2-mercaptobenzimidazole, dibutylzinc dithiocarbamates, dioctadecyl disulfide and pentaerythritol tetrakis(β-dodecylmercapto)propionate,

2-hydroxybenzophenones, such as the 4-hydroxy, 4-methoxy, 4-octyloxy, 4-decycloxy, 4-dodecyloxy, 4-benzyloxy, 4,2′,4′-trihydroxy and 2′-hydroxy-4,4′-dimethoxy derivatives,

Esters of unsubstituted and substituted benzoic acids, such as 4-tert-butylphenyl salicylate, phenyl salicylate, octylphenyl salicylate, dibenzoylresorcinol, bis(4-tert-butylbenzoyl)resorcinol, benzoylresorcinol, 2,4-di-tert-butylphenyl 3,5-di-tert-butyl-4-hydroxybenzoate, hexadecyl-3,5-di-tert-butyl-4-hydroxybenzoate, octadecyl-3,5-di-tert-butyl-4-hydroxybenzoate and 2-methyl-4,6-di-tert-butylphenyl-3,5-di-tert-butyl-4-hydroxybenzoate,

Acrylates, such as ethyl α-cyano-β,β-diphenylacrylate, isooctyl α-cyano-β,β-diphenylacrylate, methyl α-methoxycarbonylcinnamate, methyl α-cyano-β-methyl-p-methoxycinnamate, butyl-α-cyano-β-methyl-p-methoxycinnamate and methyl-α-methoxycarbonyl-p-methoxycinnamate, sterically hindered amines, such as bis(2,2,6,6-tetramethylpiperidin-4-yl)sebacate, bis(2,2,6,6-tetramethylpiperidin-4-yl)succinate, bis(1,2,2,6,6-pentamethylpiperidin-4-yl)sebacate, bis(1-octyloxy-2,2,6,6-tetramethylpiperidin-4-yl)sebacate, bis(1,2,2,6,6-pentamethylpiperidin-4-yl)-n-butyl-3,5-di-tert-butyl-4-hydroxybenzylmalonate, the condensation product of 1-(2-hydroxyethyl)-2,2,6,6-tetramethyl-4-hydroxypiperidine and succinic acid, the condensation product of N,N′-bis(2,2,6,6-tetramethylpiperidin-4-yl)hexamethylenediamine and 4-tert-octylamino-2,6-dichloro-1,3,5-triazine, tris(2,2,6,6-tetramethylpiperidin-4-yl)nitrilotriacetate, tetrakis(2,2,6,6-tetramethylpiperidin-4-yl) 1,2,3,4-butanetetracarboxylate, 1,1′-(1,2-ethylene)bis(3,3,5,5-tetramethylpiperazinone), 4-benzoyl-2,2,6,6-tetramethylpiperidine, 4-stearyloxy-2,2,6,6-tetramethylpiperidine, bis(1,2,2,6,6-pentamethylpiperidin-4-yl)2-n-butyl-2-(2-hydroxy-3,5-di-tert-butylbenzyl)malonate, 3-n-octyl-7,7,9,9-tetramethyl-1,3,8-triazaspiro[4.5]decane-2,4-dione, bis(1-octyloxy-2,2,6,6-tetramethylpiperidin-4-yl)sebacate, bis(1-octyloxy-2,2,6,6-tetramethylpiperidin-4-yl)succinate, the condensation product of N,N′-bis(2,2,6,6-tetramethylpiperidin-4-yl)hexamethylenediamine and 4-morpholino-2,6-dichloro-1,3,5-triazine, the condensation product of 2-chloro-4,6-bis(4-n-butylamino-2,2,6,6-tetramethylpiperidin-4-yl)-1,3,5-triazine and 1,2-bis(3-aminopropylamino)ethane, the condensation product of 2-chloro-4,6-di(4-n-butylamino-1,2,2,6,6-pentamethylpiperidin-4-yl)-1,3,5-triazine and 1,2-bis(3-aminopropylamino)ethane, 8-acetyl-3-dodecyl-7,7,9,9-tetramethyl-1,3,8-triazaspiro[4.5]-decane-2,4-dione, 3-dodecyl-1-(2,2,6,6-tetramethylpiperidin-4-yl)pyrrolidine-2,5-dione, 3-dodecyl-1-(1,2,2,6,6-pentamethylpiperidin-4-yl)pyrrolidine-2,5-dione, a mixture of 4-hexadecyloxy- and 4-stearyloxy-2,2,6,6-tetramethylpiperidine, the condensation product of N,N′-bis(2,2,6,6-tetramethylpiperidin-4-yl)hexamethylenediamine and 4-cyclohexylamino-2,6-dichloro-1,3,5-triazine, the condensation product of 1,2-bis(3-aminopropylamino)ethane and 2,4,6-trichloro-1,3,5-triazine, 4-butylamino-2,2,6,6-tetramethylpiperidine, N-(2,2,6,6-tetramethylpiperidin-4-yl)-n-dodecylsuccinimide, N-(1,2,2,6,6-pentamethylpiperidin-4-yl)-n-dodecylsuccinimide, 2-undecyl-7,7,9,9-tetramethyl-1-oxa-3,8-diaza-4-oxo-spiro[4.5]-decane, the condensation product of 7,7,9,9-tetramethyl-2-cycloundecyl-1-oxa-3,8-diaza-4-oxospiro-[4.5]decane and epichlorohydrin, the condensation products of 4-amino-2,2,6,6-tetramethylpiperidine with tetramethylolacetylenediureas and poly(methoxypropyl-3-oxy)-[4(2,2,6,6-tetramethyl)piperidinyl]-siloxane,

Oxalamides, such as 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxalamide, 2-ethoxy-5-tert-butyl-2′-ethoxanilide and its mixture with 2-ethoxy-2′-ethyl-5,4′-di-tert-butoxanilide, and mixtures of ortho-, para-methoxy-disubstituted oxanilides and mixtures of ortho- and para-ethoxy-disubstituted oxanilides, and

2-(2-hydroxyphenyl)-1,3,5-triazines, such as 2,4,6-tris-(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)phenyl]-4,6-bis(2,4-dimethyl)-1,3,5-triazine, 2-[2-hydroxy-4-(2-hydroxy-3-octyloxypropoxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis-(2,4-dimethylphenyl)-1,3,5-triazine, 2-(2-hydroxy-4-hexyloxyphenyl)-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine and 2-(2-hydroxyphenyl)-4-(4-methoxyphenyl)-6-phenyl-1,3,5-triazine.

In another preferred embodiment the RM formulation comprises one or more solvents, which are preferably selected from organic solvents. The solvents are preferably selected from ketones such as acetone, methyl ethyl ketone, methyl propyl ketone, methyl isobutyl ketone or cyclohexanone; acetates such as methyl, ethyl or butyl acetate or methyl acetoacetate; alcohols such as methanol, ethanol or isopropyl alcohol; aromatic solvents such as toluene or xylene; alicyclic hydrocarbons such as cyclopentane or cyclohexane; halogenated hydrocarbons such as di- or trichloromethane; glycols or their esters such as PGMEA (propyl glycol monomethyl ether acetate), γ-butyrolactone. It is also possible to use binary, ternary or higher mixtures of the above solvents.

In case the RM formulation contains one or more solvents, the total concentration of all solids, including the RMs, in the solvent(s) is preferably from 10 to 60%.

Polymerisation of the RMs is preferably carried out in the presence of an initiator absorbing at the wavelength of the actinic radiation. For this purpose, preferably the RM formulation contains one or more polymerisation initiators.

For example, when polymerising by means of UV light, a photoinitiator can be used that decomposes under UV irradiation to produce free radicals or ions that start the polymerisation reaction. For polymerising acrylate or methacrylate groups preferably a radical photoinitiator is used. For polymerising vinyl, epoxide or oxetane groups preferably a cationic photoinitiator is used. It is also possible to use a thermal polymerisation initiator that decomposes when heated to produce free radicals or ions that start the polymerisation. Typical radical photoinitiators are for example the commercially available Irgacure® or Darocure® (Ciba AG). for example Irgacure 127, Irgacure 184, Irgacure 369, Irgacure 651, Irgacure 817, Irgacure 907, Irgacure 1300, Irgacure, Irgacure 2022, Irgacure 2100, Irgacure 2959, or Darcure TPO. Preferably The RM formulation preferably comprises a combination of one or more, preferably of one or two of such photoinitiators.

A typical cationic photoinitiator is for example UVI 6974 (Union Carbide).

The concentration of the polymerisation initiator(s) as a whole in the RM formulation is preferably from 0.1 to 10%, very preferably from 0.5 to 8%, more preferably 2 to 6%.

In particular the RM formulation comprises:
1 to 80%, preferably 30 to 70% of compounds of formula I,
1 to 60%, preferably 5 to 40% of di- or multireactive RMs, preferably selected of one or more compounds of formula DRM,
optionally 1 to 80%, preferably 5 to 20% of monoreactive RMs preferably selected of one or more compounds of formula MRM,
optionally, 0.1 to 10%, preferably 0.5 to 8%, more preferably 2 to 6% of one or more polymerisation initiators,
optionally, 0.01 to 5%, preferably 0.01 to 1% of one or more surfactants,
optionally, 1 to 10%, preferably 2 to 6% of one or more chiral compounds, preferably selected from one or more compounds or formulae C-1 to C-III and/or CRM.

The preparation of polymers according to this invention can be carried out by methods that are known to the skilled person and described in the literature, for example in D. J. Broer; G. Challa; G. N. Mol, Macromol. Chem, 1991, 192, 59.

Typically the RM, RM mixture or RM formulation is coated or otherwise applied onto a substrate, for example by a coating or printing method, where the RMs are aligned into uniform orientation. Preferably the RMs are aligned into planar alignment, i.e. with the long molecular axes of the RM molecules aligned parallel to the substrate. However it is likewise preferred to align the RMs into a homeotropic alignment or into a tilted alignment.

The aligned RMs are then polymerised in situ, preferably at a temperature where they exhibit an LC phase, for example by exposure to heat or actinic radiation. Preferably the RMs are polymerised by photo-polymerisation, very preferably by UV-photopolymerisation, to fix the uniform alignment. If necessary, uniform alignment can be promoted by additional means like shearing or annealing of the RMs, surface treatment of the substrate, or adding surfactants to the RM mixture or the RM formulation.

As substrate for example glass or quartz sheets or plastic films can be used. It is also possible to put a second substrate on top of the coated material prior to and/or during and/or after polymerisation. The substrates can be removed after polymerisation or not. When using two substrates in case of curing by actinic radiation, at least one substrate has to be transmissive for the actinic radiation used for the polymerisation. Isotropic or birefringent substrates can be used. In case the substrate is not removed from the polymerised film after polymerisation, preferably isotropic substrates are used.

Suitable and preferred plastic substrates are for example films of polyester such as polyethyleneterephthalate (PET) or polyethylene-naphthalate (PEN), polyvinylalcohol (PVA), polycarbonate (PC) or triacetylcellulose (TAC), very preferably PET or TAC films. As birefringent substrates for example uniaxially stretched plastics film can be used. PET films are commercially available for example from DuPont Teijin Films under the trade name Melinex®.

Preferably the RMs and the other solid additives are dissolved in a solvent. The solution is then coated or printed onto the substrate, for example by spin-coating or printing or other known techniques, and the solvent is evaporated off before polymerisation. In many cases it is suitable to heat the coated solution in order to facilitate the evaporation of the solvent.

The RM formulation can be applied onto the substrate by conventional coating techniques like spin-coating or blade coating. It can also be applied to the substrate by conventional printing techniques which are known to the expert, like for example screen printing, offset printing, reel-to-reel printing, letter press printing, gravure printing, rotogravure printing, flexographic printing, intaglio printing, pad printing, heat-seal printing, ink-jet printing or printing by means of a stamp or printing plate.

The RM formulation preferably exhibits planar alignment. This can be achieved for example by rubbing treatment of the substrate, by shearing the material during or after coating, by annealing the material before polymerisation, by application of an alignment layer, by applying a magnetic or electric field to the coated material, or by the addition of surface-active compounds to the formulation. Reviews of alignment techniques are given for example by I. Sage in “Thermotropic Liquid Crystals”, edited by G. W. Gray, John Wiley & Sons, 1987, pages 75-77; and by T. Uchida and H. Seki in “Liquid Crystals—Applications and Uses Vol. 3”, edited by B. Bahadur, World Scientific Publishing, Singapore 1992, pages 1-63. A review of alignment materials and techniques is given by J. Cognard, Mol. Cryst. Liq. Cryst. 78, Supplement 1 (1981), pages 1-77.

It is also possible to apply an alignment layer onto the substrate and provide the RM mixture or RM formulation onto this alignment layer. Suitable alignment layers are known in the art, like for example rubbed polyimide or alignment layers prepared by photoalignment as described in U.S. Pat. No. 5,602,661, U.S. Pat. No. 5,389,698 or U.S. Pat. No. 6,717,644.

It is also possible to induce or improve alignment by annealing the RMs at elevated temperature, but below their clearing temperature, prior to polymerisation.

Polymerisation is achieved for example by exposing the polymerisable material to heat or actinic radiation. Actinic radiation means irradiation with light, like UV light, IR light or visible light, irradiation with X-rays or gamma rays or irradiation with high energy particles, such as ions or electrons.

Preferably polymerisation is carried out by UV irradiation. As a source for actinic radiation for example a single UV lamp or a set of UV lamps can be used. When using a high lamp power the curing time can be reduced. Another possible source for actinic radiation is a laser, like for example a UV, IR or visible laser.

The curing time depends, inter alia, on the reactivity of the RMs, the thickness of the coated layer, the type of polymerisation initiator and the power of the UV lamp. The curing time is preferably ≤5 minutes, very preferably ≤3 minutes, most preferably ≤1 minute. For mass production short curing times of ≤30 seconds are preferred.

The polymerisation process is not limited to one curing step. It is also possible to carry out polymerisation by two or more steps, in which the film is exposed to two or more lamps of the same type, or two or more different lamps in sequence. The curing temperature of different curing steps might be the same or different. The lamp power and dose from different lamps might also be the same or different. In addition to the conditions described above, the process steps may also include a heat step between exposure to different lamps, as described for example in JP 2005-345982 A and JP 2005-265896 A.

Preferably polymerisation is carried out in air, but polymerising in an inert gas atmosphere like nitrogen or argon is also possible.

The thickness of a polymer film according to the present invention is preferably less than 15 microns, very preferably less than 12 microns most preferably less than 10 microns.

The RMs, RM mixtures, RM formulations and polymers of the present invention can be used in optical, electro optical or electronic devices or components thereof.

For example, they can be used in optical retardation films, polarizers, compensators, beam splitters, reflective films, alignment layers, color filters, antistatic protection sheets, or electromagnetic interference protection sheets, polarization controlled lenses for autostereoscopic 3D displays, RM lenses and IR reflection films for window applications.

The RMs, RM mixtures, RM formulations, polymers and device components of the present invention can be used for example in devices selected from electro optical displays, especially liquid crystal displays (LCDs), autostereoscopic 3D displays, organic light emitting diodes (OLEDs), optical data storage devices, and window applications.

The RMs, RM mixtures, RM formulations, polymers and device components of the present invention can be used outside the switchable LC cell of an LCD or between the substrates, usually glass substrates, forming the switchable LC cell and containing the switchable LC medium (incell application).

The RMs, RM mixtures, RM formulations, polymers and device components of the present invention can be used in conventional LC displays, for example displays with vertical alignment like the DAP (deformation of aligned phases), ECB (electrically controlled birefringence), CSH (colour super homeotropic), VA (vertically aligned), VAN or VAC (vertically aligned nematic or cholesteric), MVA (multi-domain vertically aligned), PVA (patterned vertically aligned) or PSVA (polymer stabilised vertically aligned) mode; displays with bend or hybrid alignment like the OCB (optically compensated bend cell or optically compensated birefringence), R-OCB (reflective OCB), HAN (hybrid aligned nematic) or pi-cell (π-cell) mode; displays with twisted alignment like the TN (twisted nematic), HTN (highly twisted nematic), STN (super twisted nematic), AMD-TN (active matrix driven TN) mode; displays of the IPS (in plane switching) mode, or displays with switching in an optically isotropic phase.

The RMs, RM mixtures, RM formulations and polymers of the present invention can be used for various types of optical films, like twisted optical retarders, reflective polarisers and brightness enhancement films.

Above and below, percentages are percent by weight unless stated otherwise. All temperatures are given in degrees Celsius. m.p. denotes melting point, cl.p. denotes clearing point, T_{g} denotes glass transition temperature. Furthermore, C=crystalline state, N=nematic phase, S=smectic phase and I=isotropic phase. The data between these symbols represent the transition temperatures. Δn denotes the optical anisotropy or birefringence (Δn=nₑ−nₒ, where nₒ denotes the refractive index perpendicular to the longitudinal molecular axes and nₑ denotes the refractive index parallel thereto), measured at 589 nm and 20° C. The optical and electro optical data are measured at 20° C., unless expressly stated otherwise. “Clearing point” and “clearing temperature” mean the temperature of the transition from an LC phase into the isotropic phase.

Unless stated otherwise, the percentages of solid components in an RM mixture or RM formulation as described above and below refer to the total amount of solids in the mixture or formulation, i.e. without any solvents.

Unless stated otherwise, all optical, electro optical properties and physical parameters like birefringence, permittivity, electrical conductivity, electrical resistivity and sheet resistance, refer to a temperature of 20° C.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

For the present invention,

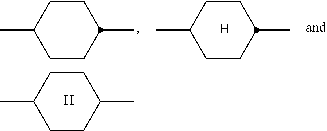

denote trans-1,4-cyclohexylene, and

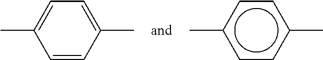

denote 1,4-phenylene.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

The following examples are intended to explain the invention without restricting it. The methods, structures and properties described hereinafter can also be applied or transferred to materials that are claimed in this invention but not explicitly described in the foregoing specification or in the examples.

### Examples

### Compound Example 1

Compound (RM-1) was prepared as described below.

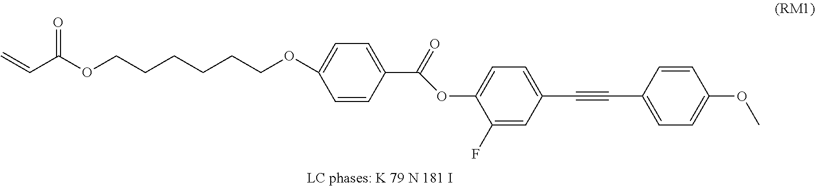 [SMILES: C=CC(=O)OCCCCCCOc1ccc(C(=O)Oc2ccc(C#Cc3ccc(OC)cc3)cc2F)cc1]

### Stage 1

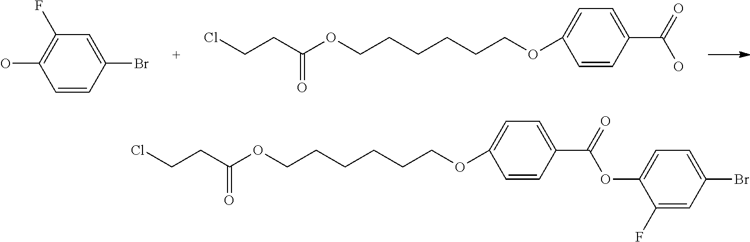 [SMILES: Oc1ccc(Br)cc1F ; O=C(CCCl)OCCCCCCOc1ccc(C(=O)O)cc1 ; O=C(CCCl)OCCCCCCOc1ccc(C(=O)Oc2ccc(Br)cc2F)cc1]

To a stirred solution of 4-bromo-2-fluorophenol (10 g, 52.4 mmol), HHBA-3-chloropropionate (17.21 g, 52.4 mmol) and 4-dimethyaminopyridine (0.2 g, 1.6 mmol) in dry dichloromethane (100 ml) is added 1M N,N-dicyclohexylcarbodiimide in dichloromethane (55 ml, 55 mmol). The mixture is stirred for 16 hours and then concentrated under reduced pressure. dichloromethane (10 ml) is added and the mixture applied to a column of silica eluting with dichloromethane. Appropriate fractions are combined and concentrated to give an oil that was crystallised from petroleum ether 40/60 (22.96 g, 87.3%).

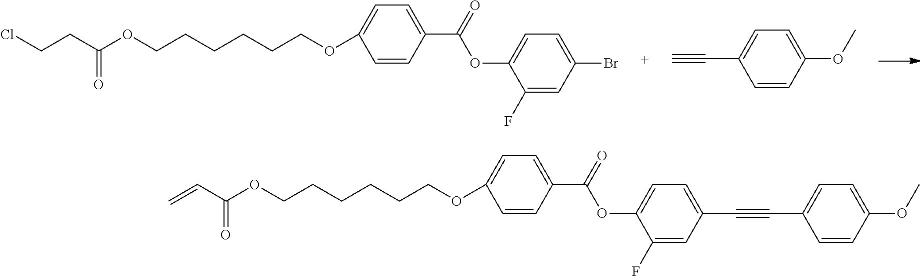

In a 500 ml 3 necked round bottom flask under nitrogen is placed the product of stage 1 (5.01 g, 10 mmol), 4-ethynylanisole (1.32 g, 10 mmol) and diisopropylamine (50 ml). The flask is flushed with nitrogen, ultrasonicated for 30 minutes and again flushed with more nitrogen. Pd(OAc)₂ (133 mg, 0.59 mmol), copper(I) iodide (66.6 mg, 0.3 mmol) and tri-tert-butylphosphonium tetrafluoroborate (150 mg, 0.52 mmol) are added to the mixture, which is then heated to 85° C. for 1 hour. The mixture is cooled and the solids filtered off and washed through with CH₂Cl₂. Concentration of the filtrate under reduced pressure gives a dark colored solid that is dissolved in a minimum amount of CH₂Cl₂ (10 ml) and purified on a column of silica eluted CH₂Cl₂. Concentration under reduced pressure gives an oil that is crystallized from ethanol, then re-crystallized from acetonitrile (3.87 g, 64.9%).

### Compound Examples 2-8

The following compounds are prepared in analogy to the synthesis described in Example 1.

| No. | Structure | LC phase |
|---|---|---|
| RM-II | 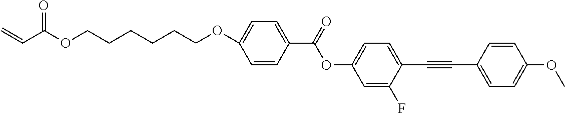 | K 91.6 N 187 I |
| RM-III | 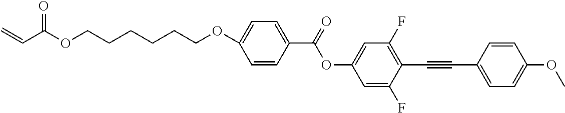 [SMILES: C=CC(=O)OCCCCCCOc1ccc(C(=O)Oc2cc(F)c(C#Cc3ccc(OC)cc3)c(F)c2)cc1] | K 91.7 N 175 I |
| RM-IV | 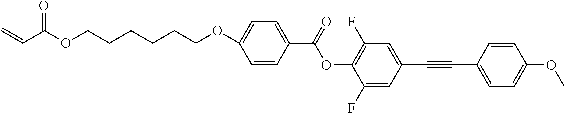 | — |
| RM-V | 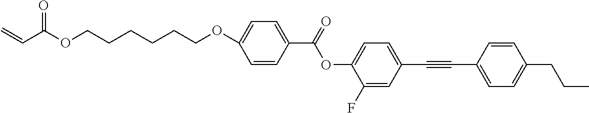 | K 67.9 N 156 I |
| RM-VI | 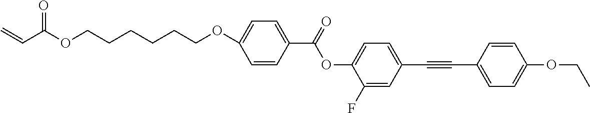 | K 67.3 N 189 I |
| RM-VII | 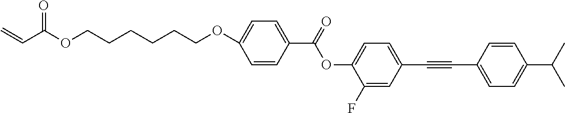 [SMILES: C=CC(=O)OCCCCCCOc1ccc(C(=O)Oc2ccc(C#Cc3ccc(C(C)C)cc3)cc2F)cc1] | K 80.1 N 119 I |
| RM-VIII | 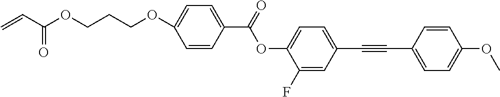 [SMILES: C=CC(=O)OCCCOc1ccc(C(=O)Oc2ccc(C#Cc3ccc(OC)cc3)cc2F)cc1] | K 102 N 209 I |
| RM-IX | 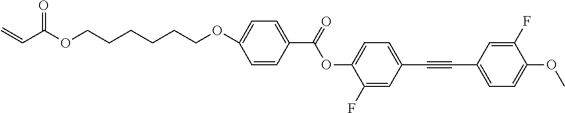 | — |
| RM-X | 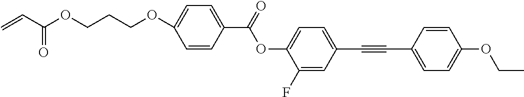 | — |

### Comparison Example 1

Compound (C1), compound (C2) and compound (C3) are prepared in analogy to the synthesis described in Example 1.

| No. | Structure | LC phase |
|---|---|---|
| A | 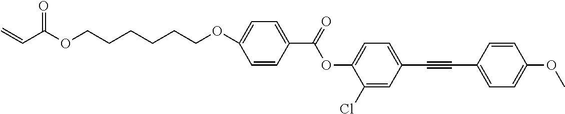 [SMILES: C=CC(=O)OCCCCCCOc1ccc(C(=O)Oc2ccc(C#Cc3ccc(OC)cc3)cc2Cl)cc1] | K 66.9 N 158 I |
| B | 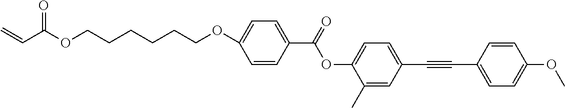 [SMILES: C=CC(=O)OCCCCCCOc1ccc(C(=O)Oc2ccc(C#Cc3ccc(OC)cc3)cc2C)cc1] | K 82.9 N 161 I |
| C | 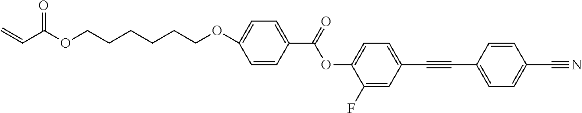 [SMILES: C=CC(=O)OCCCCCCOc1ccc(C(=O)Oc2ccc(C#Cc3ccc(C#N)cc3)cc2F)cc1] | K 87.3 N 202 I |

### Yellowing

UV-Vis spectroscopy was used to measure the compound yellowing by measuring the percentage transmission for each of the compounds across the visible range. This was done by dissolving 1 wt. % of each compound in a solvent, usually dichloromethane, and measuring the solutions percentage transmission on the Hitachi UV-Vis spectrometer, with air as a baseline. The solutions were then cured at a variety of different doses (0, 100, 500, 1000 and 3000 mJ) and the transmission measured again.

Anhydrous dichloromethane was used to dissolve the mixtures, as it remains unaffected when exposed to UV light. By comparing these percentage transmissions it could be concluded, which compound yellow and to what extent.

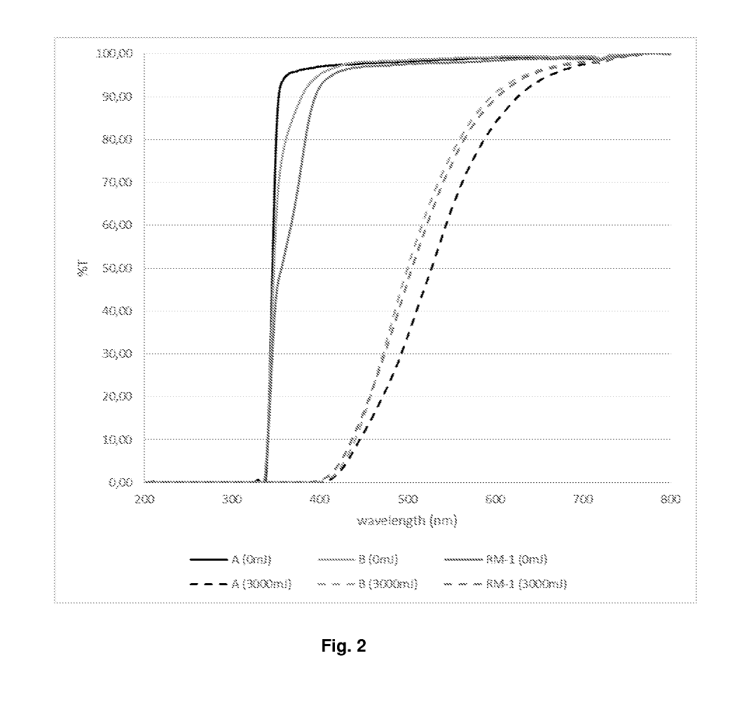

FIG. 2 show the results of yellowing studies for compound RM-1 of the invention in comparison to compound A and compound B of prior art.

From FIG. 2 can be seen that RM-1 and A-show the least amount of change in yellowing when exposed to UV-light. In comparison, compound B of prior art, shows a significant increase in yellowing.

### Mixture Examples

The following mixtures are prepared:

### Comparison Example 1: Mixture C-1

| Compound | Conc. |
|---|---|
| 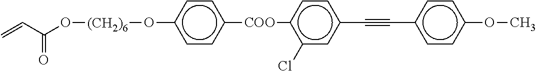 | 49.005 |
| 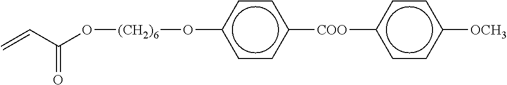 | 9.910 |
| 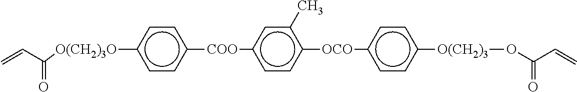 | 22.400 |
| 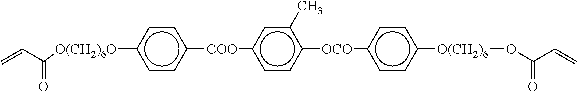 | 5.600 |
| 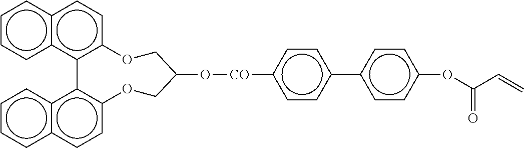 | 3.250 |
| 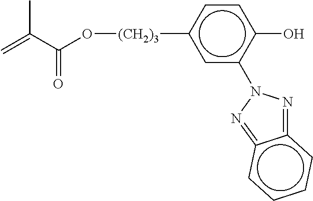 | 2.600 |
| 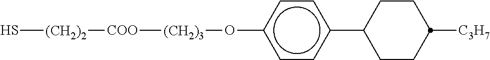 | 2.000 |
| Irgacure 819 | 0.240 |
| Irgacure 907 | 4.890 |
| Irganox 1076 | 0.080 |
| Tego Rad 2500 | 0.025 |

The clearing point of comparison mixture C-1 is 90.9° C. Comparison Example 2: Mixture C-2

| Compound | Conc. |
|---|---|
| 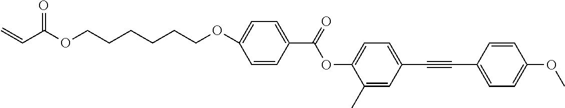 [SMILES: C=CC(=O)OCCCCCCOc1ccc(C(=O)Oc2ccc(C#Cc3ccc(OC)cc3)cc2C)cc1] | 49.005 |
| 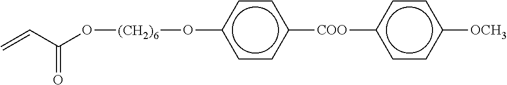 | 9.910 |
| 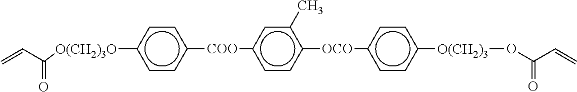 | 22.400 |
| 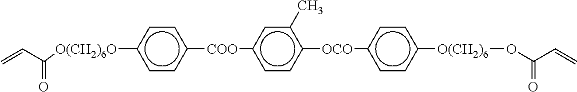 | 5.600 |
| 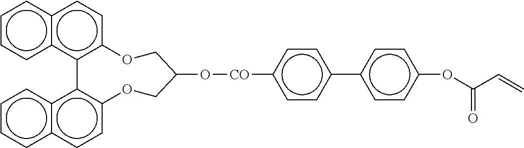 | 3.250 |
| 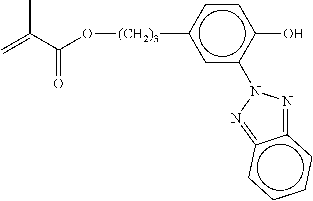 | 2.600 |
| 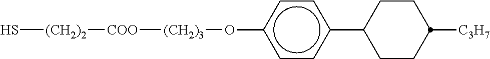 | 2.000 |
| Irgacure 819 | 0.240 |
| Irgacure 907 | 4.890 |
| Irganox 1076 | 0.080 |
| Tego Rad 2500 | 0.025 |

The clearing point of comparison mixture C-2 is 93.3° C.

### Comparison Example: Mixture C-3

| Compound | Conc. |
|---|---|
| 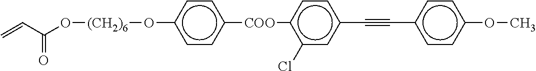 | 66.075 |
| 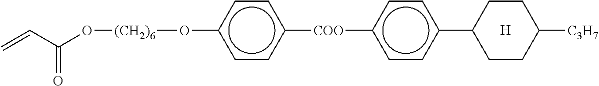 | 11.020 |
| 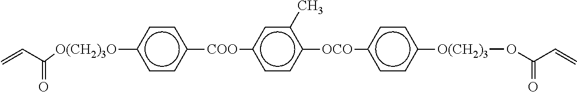 | 8.280 |
| 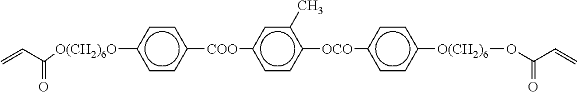 | 1.990 |
| 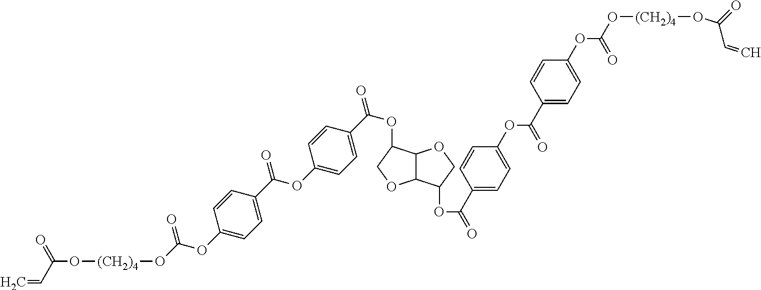 | 5.440 |
| 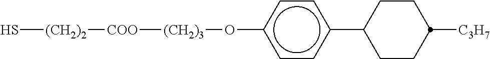 | 1.960 |
| Irgacure 369 | 4.890 |
| Darocur TPO | 0.240 |
| lrganox 1076 | 0.080 |
| Tego Twin 4000 | 0.025 |

The clearing point of comparison mixture C-3 is 123.5° C.

### Comparison Example: Mixture C-4

| Compound | Conc. |
|---|---|
| 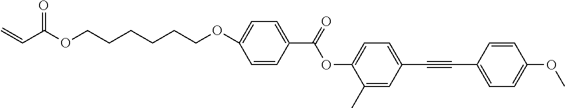 [SMILES: C=CC(=O)OCCCCCCOc1ccc(C(=O)Oc2ccc(C#Cc3ccc(OC)cc3)cc2C)cc1] | 66.075 |
| 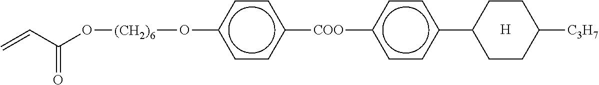 | 11.020 |
| 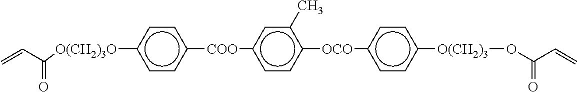 | 8.280 |
| 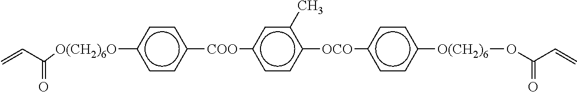 | 1.990 |
| 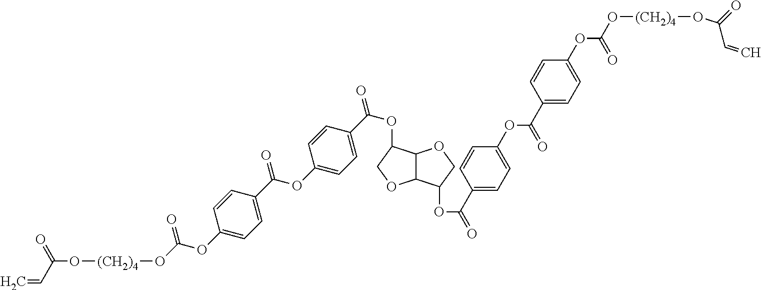 | 5.440 |
| 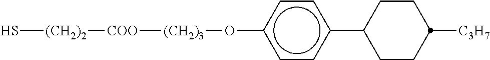 | 1.960 |
| Irgacure 369 | 4.890 |
| Darocur TPO | 0.240 |
| Irganox 1076 | 0.080 |
| Tego Twin 4000 | 0.025 |

The clearing point of comparison mixture C-3 is 124.9° C.

### Mixture Example 1: Mixture M-1

| Compound | Conc. |
|---|---|
| 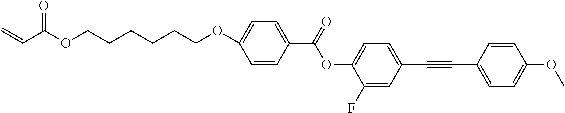 [SMILES: C=CC(=O)OCCCCCCOc1ccc(C(=O)Oc2ccc(C#Cc3ccc(OC)cc3)cc2F)cc1] | 49.005 |
| 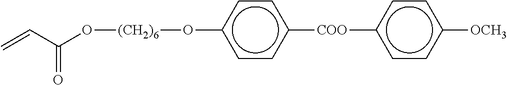 | 9.910 |
| 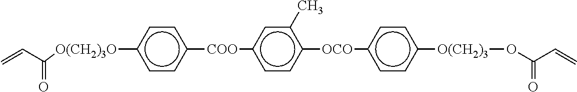 | 22.400 |
| 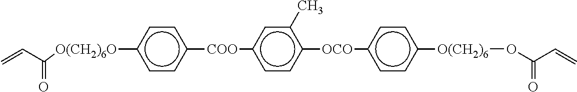 | 5.600 |
| 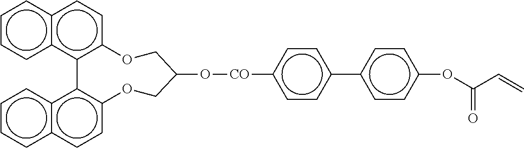 | 3.250 |
| 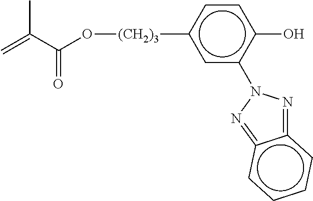 | 2.600 |
| 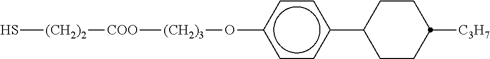 | 2.000 |
| Irgacure 819 | 0.240 |
| Irgacure 907 | 4.890 |
| Irganox 1076 | 0.080 |
| Tego Rad 2500 | 0.025 |

The clearing point of mixture M-1 is 106.5° C.

### Mixture Example 2: Mixture M-2

| Compound | Conc. |
|---|---|
| 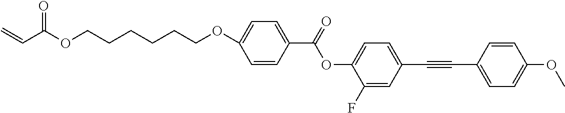 [SMILES: C=CC(=O)OCCCCCCOc1ccc(C(=O)Oc2ccc(C#Cc3ccc(OC)cc3)cc2F)cc1] | 67.745 |
| 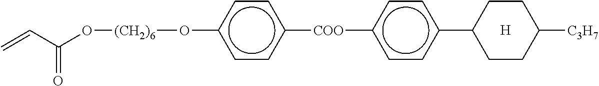 | 11.3 |
| 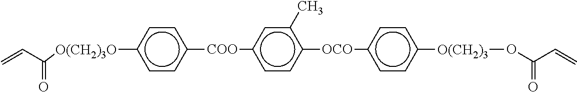 | 8.49 |
| 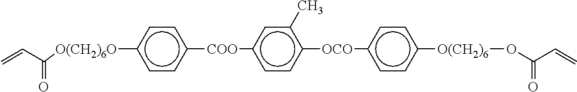 | 2.04 |
| 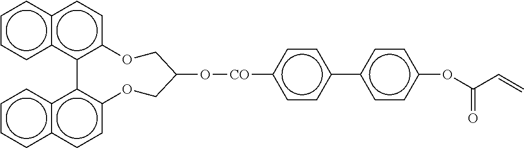 | 3.23 |
| 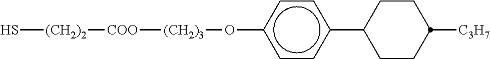 | 1.96 |
| Irgacure 907 | 4.89 |
| Irgacure 819 | 0.24 |
| Irganox 1076 | 0.08 |
| Tego Rad 2500 | 0.025 |

### Mixture Example 3: Mixture M-3

| Compound | Conc. |
|---|---|
| 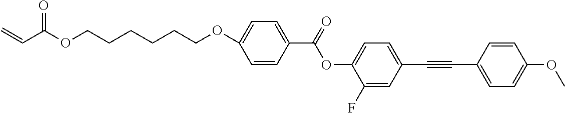 [SMILES: C=CC(=O)OCCCCCCOc1ccc(C(=O)Oc2ccc(C#Cc3ccc(OC)cc3)cc2F)cc1] | 67.745 |
| 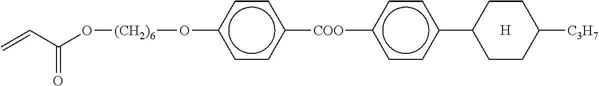 | 11.3 |
| 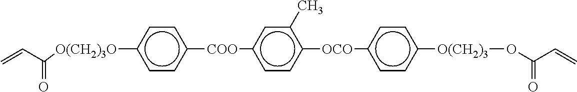 | 8.49 |
| 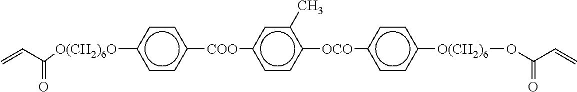 | 2.04 |
| 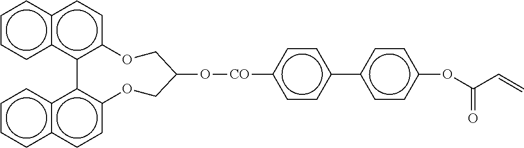 | 3.23 |
| 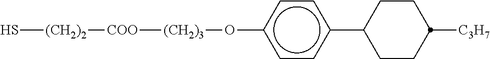 | 1.96 |
| Irgacure 907 | 4.89 |
| Irgacure 819 | 0.24 |
| Irganox 1076 | 0.08 |
| Tego Twin 4000 | 0.025 |

### Mixture Example 4: Mixture M-4

| Compound | Conc. |
|---|---|
| 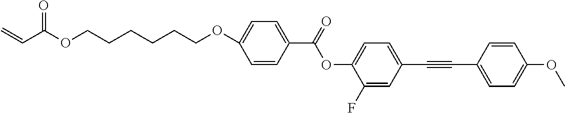 [SMILES: C=CC(=O)OCCCCCCOc1ccc(C(=O)Oc2ccc(C#Cc3ccc(OC)cc3)cc2F)cc1] | 67.745 |
| 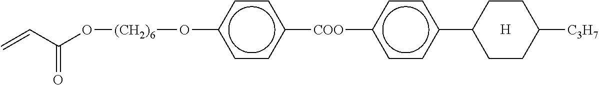 | 11.3 |
| 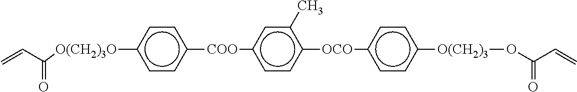 | 8.49 |
| 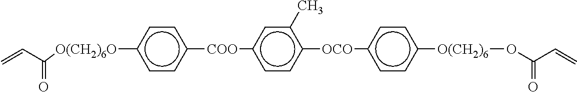 | 2.04 |
| 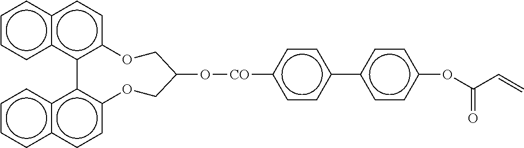 | 3.23 |
| 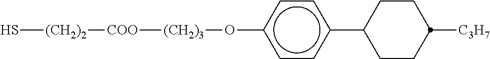 | 1.96 |
| Irgacure 907 | 4.89 |
| Irgacure 819 | 0.24 |
| Irganox 1076 | 0.08 |
| VP-A 146 (surfactant) | 0.025 |

### Mixture Example 5: Mixture M-5

| Compound | Conc. |
|---|---|
| 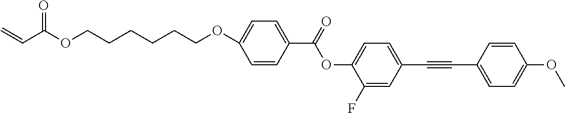 [SMILES: C=CC(=O)OCCCCCCOc1ccc(C(=O)Oc2ccc(C#Cc3ccc(OC)cc3)cc2F)cc1] | 67.745 |
| 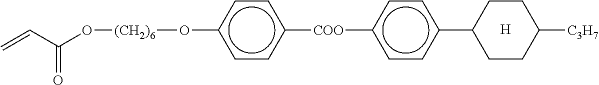 | 11.3 |
| 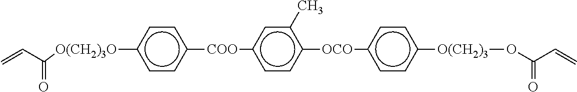 | 8.49 |
| 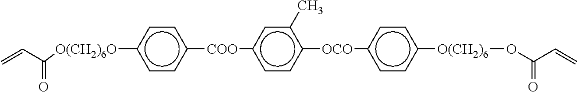 | 2.04 |
| 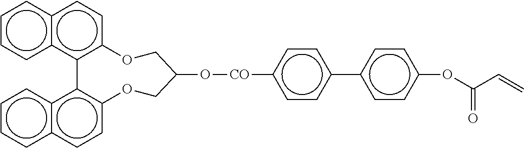 | 3.23 |
| 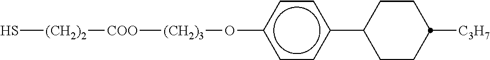 | 1.96 |
| Irgacure 907 | 4.89 |
| Irgacure 819 | 0.24 |
| Irganox 1076 | 0.08 |
| Tego Airex 936 | 0.025 |

### Mixture Example 6: Mixture M-6

| Compound | Conc. |
|---|---|
| 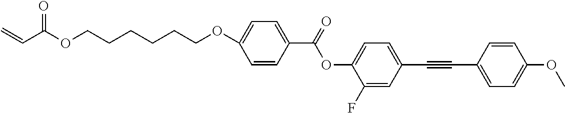 [SMILES: C=CC(=O)OCCCCCCOc1ccc(C(=O)Oc2ccc(C#Cc3ccc(OC)cc3)cc2F)cc1] | 49.005 |
| 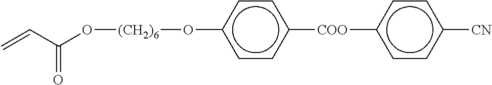 | 9.910 |
| 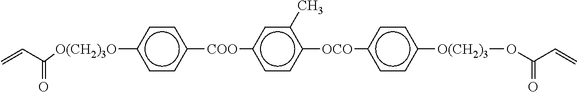 | 22.400 |
| 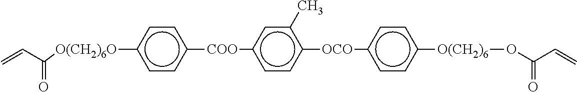 | 5.600 |
| 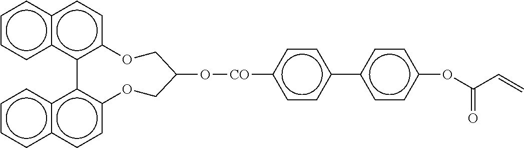 | 3.250 |
| 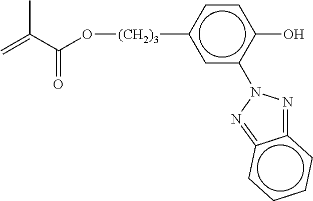 | 2.600 |
| 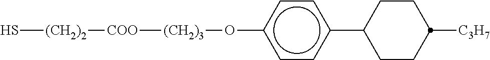 | 2.000 |
| Irgacure 819 | 0.240 |
| Irgacure 907 | 4.890 |
| Irganox 1076 | 0.080 |
| Tego Rad 2500 | 0.025 |

### Mixture Example 7: Mixture M-7

| Compound | Conc. |
|---|---|
| 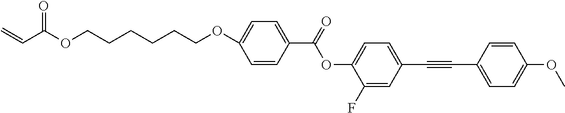 [SMILES: C=CC(=O)OCCCCCCOc1ccc(C(=O)Oc2ccc(C#Cc3ccc(OC)cc3)cc2F)cc1] | 48.99 |
| 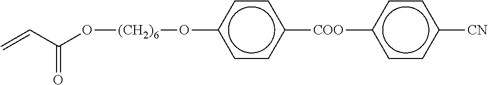 | 9.910 |
| 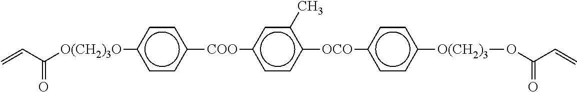 | 22.400 |
| 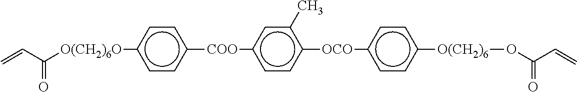 | 5.600 |
| 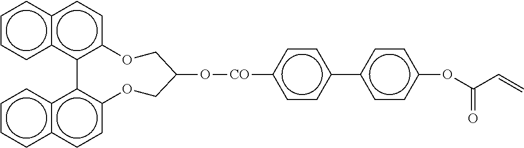 | 3.250 |
| 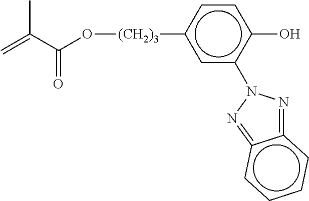 | 2.600 |
| 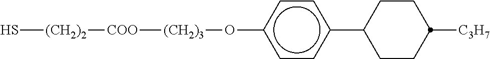 | 2.000 |
| Irgacure 819 | 0.240 |
| Irgacure 907 | 4.890 |
| Irganox 1076 | 0.080 |
| Tego Rad 2500 | 0.04 |

### Mixture Example 8: Mixture M-8

| Compound | Conc. |
|---|---|
| 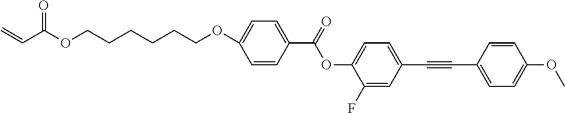 [SMILES: C=CC(=O)OCCCCCCOc1ccc(C(=O)Oc2ccc(C#Cc3ccc(OC)cc3)cc2F)cc1] | 49.02 |
| 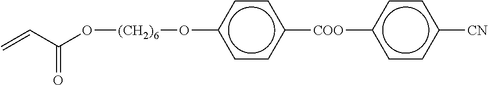 | 9.910 |
| 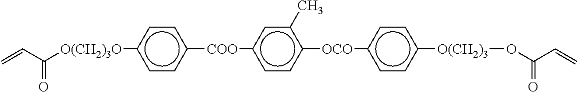 | 22.400 |
| 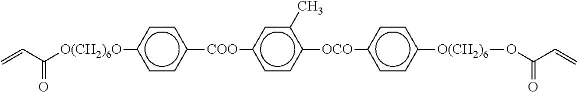 | 5.600 |
| 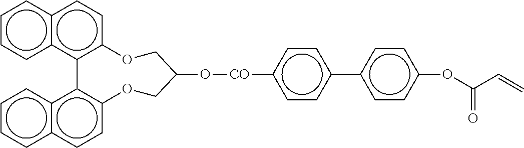 | 3.250 |
| 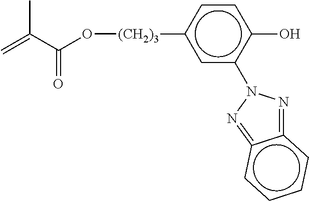 | 2.600 |
| 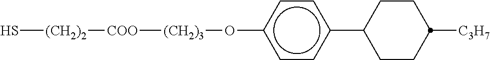 | 2.000 |
| Irgacure 819 | 0.240 |
| Irgacure 907 | 4.890 |
| Irganox 1076 | 0.080 |
| Tego Rad 2500 | 0.01 |

### Mixture Example 9: Mixture M-9

| Compound | Conc. |
|---|---|
| 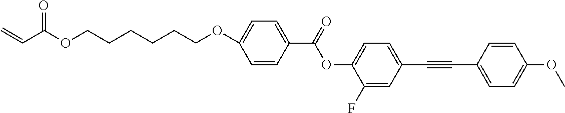 [SMILES: C=CC(=O)OCCCCCCOc1ccc(C(=O)Oc2ccc(C#Cc3ccc(OC)cc3)cc2F)cc1] | 49.005 |
| 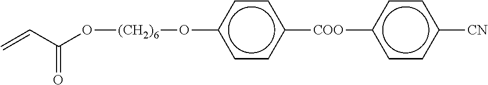 | 9.910 |
| 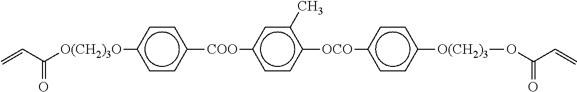 | 22.400 |
| 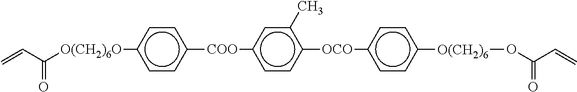 | 5.600 |
| 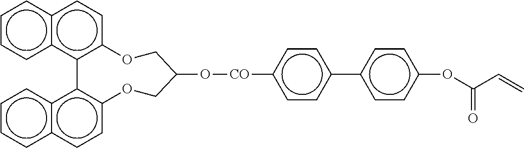 | 3.250 |
| 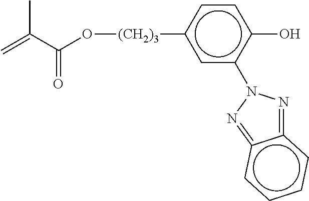 | 2.600 |
| 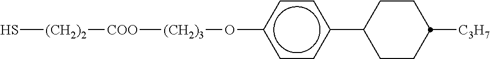 | 2.000 |
| Irgacure 819 | 0.240 |
| Irgacure 907 | 4.890 |
| Irganox 1076 | 0.080 |
| Tego Rad 2500 | 0.025 |

### Mixture Example 10: Mixture M-10

| Compound | Conc. |
|---|---|
| 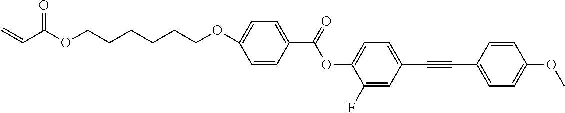 [SMILES: C=CC(=O)OCCCCCCOc1ccc(C(=O)Oc2ccc(C#Cc3ccc(OC)cc3)cc2F)cc1] | 47.765 |
| 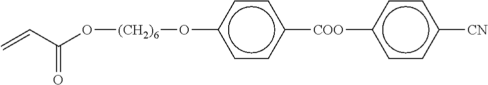 | 9.66 |
| 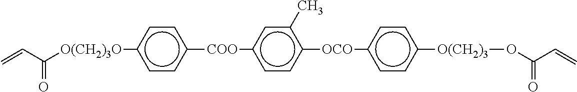 | 21.84 |
| 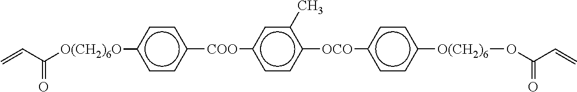 | 5.46 |
| 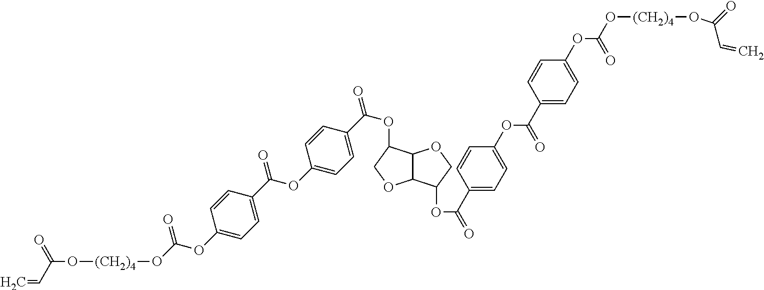 | 5.44 |
| 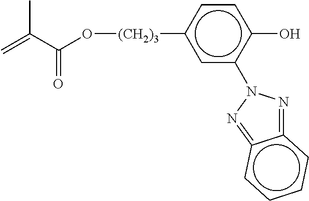 | 2.600 |
| 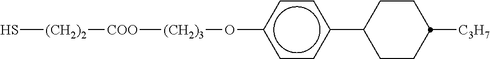 | 2.000 |
| Irgacure 819 | 0.240 |
| Irgacure 907 | 4.890 |
| Irganox 1076 | 0.080 |
| Tego Rad 2500 | 0.025 |

### Mixture Example 11: Mixture M-11

| Compound | Conc. |
|---|---|
| 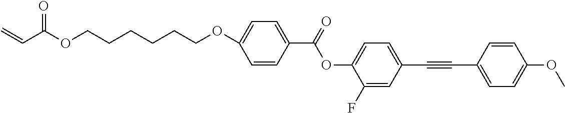 [SMILES: C=CC(=O)OCCCCCCOc1ccc(C(=O)Oc2ccc(C#Cc3ccc(OC)cc3)cc2F)cc1] | 48.605 |
| 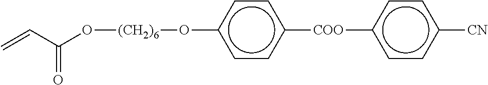 | 9.83 |
| 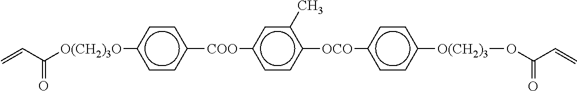 | 22.22 |
| 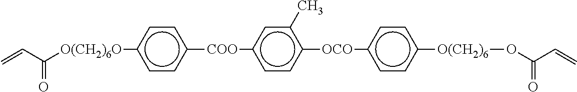 | 5.56 |
| 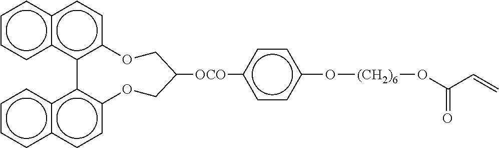 | 3.95 |
| 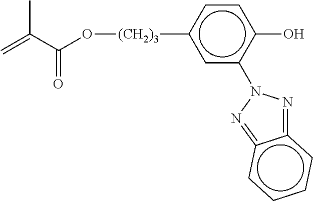 | 2.600 |
| 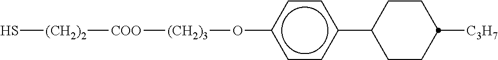 | 2.000 |
| Irgacure 819 | 0.240 |
| Irgacure 907 | 4.890 |
| Irganox 1076 | 0.080 |
| Tego Rad 2500 | 0.025 |

### Mixture Example 12: Mixture M-12

| Compound | Conc. |
|---|---|
| 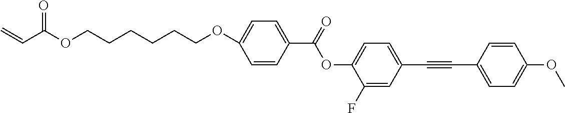 [SMILES: C=CC(=O)OCCCCCCOc1ccc(C(=O)Oc2ccc(C#Cc3ccc(OC)cc3)cc2F)cc1] | 49.325 |
| 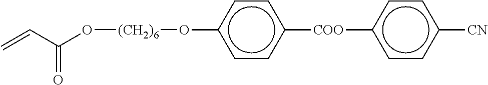 | 9.98 |
| 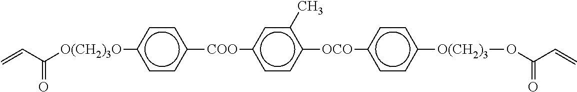 | 22.55 |
| 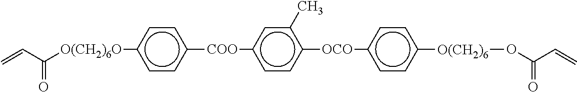 | 5.64 |
| R-5011 | 2.67 |
| 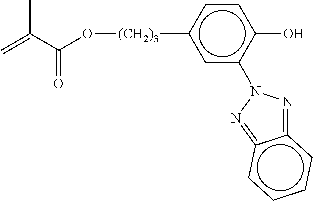 | 2.600 |
| 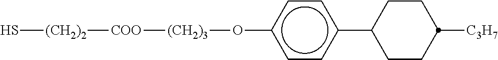 | 2.000 |
| Irgacure 819 | 0.240 |
| Irgacure 907 | 4.890 |
| Irganox 1076 | 0.080 |
| Tego Rad 2500 | 0.025 |

### Mixture Example 13: Mixture M-13

| Compound | Conc. |
|---|---|
| 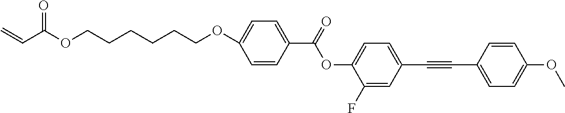 [SMILES: C=CC(=O)OCCCCCCOc1ccc(C(=O)Oc2ccc(C#Cc3ccc(OC)cc3)cc2F)cc1] | 49.005 |
| 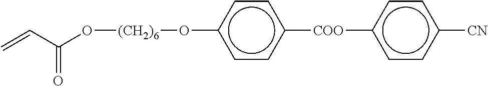 | 9.91 |
| 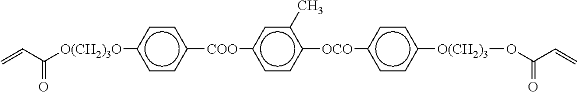 | 22.4 |
| 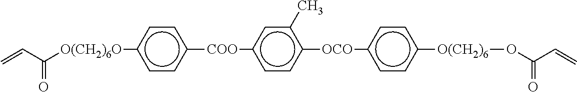 | 5.6 |
| 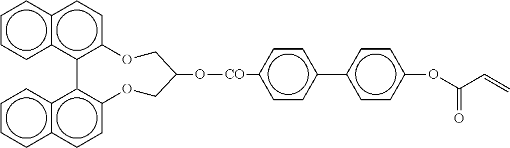 | 3.25 |
| 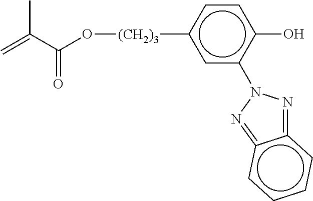 | 2.600 |
| 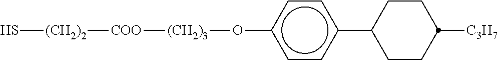 | 2.000 |
| Irgacure 2022 | 0.240 |
| Irgacure 369 | 4.890 |
| Irganox 1076 | 0.080 |
| Tego Rad 2500 | 0.025 |

### Mixture Example 14: Mixture M-14

| Compound | Conc. |
|---|---|
| 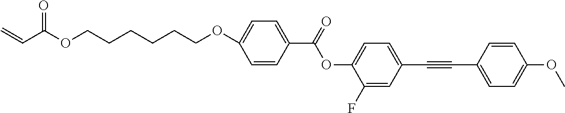 [SMILES: C=CC(=O)OCCCCCCOc1ccc(C(=O)Oc2ccc(C#Cc3ccc(OC)cc3)cc2F)cc1] | 49.005 |
| 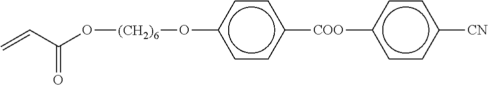 | 9.91 |
| 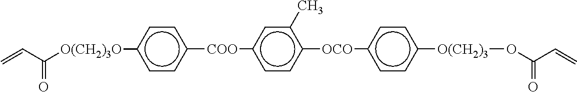 | 22.4 |
| 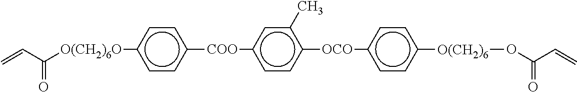 | 5.6 |
| 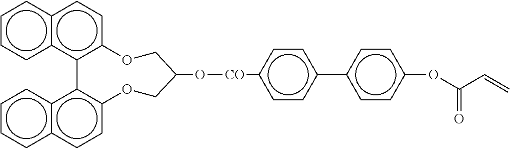 | 3.25 |
| 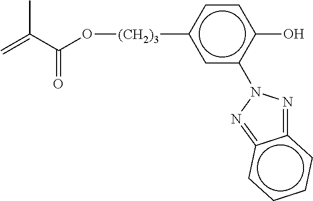 | 2.600 |
| 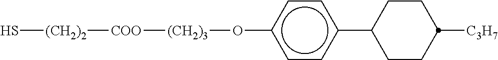 | 2.000 |
| Irgacure 2100 | 0.240 |
| Irgacure 1300 | 4.890 |
| Irganox 1076 | 0.080 |
| Tego Rad 2500 | 0.025 |

### Mixture Example 15: Mixture M-15

| Compound | Conc. |
|---|---|
| 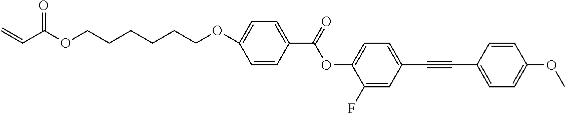 [SMILES: C=CC(=O)OCCCCCCOc1ccc(C(=O)Oc2ccc(C#Cc3ccc(OC)cc3)cc2F)cc1] | 49.005 |
| 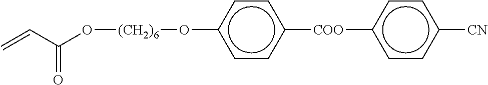 | 9.91 |
| 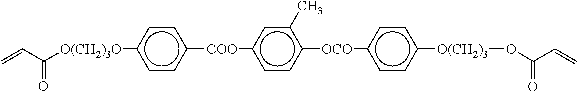 | 22.4 |
| 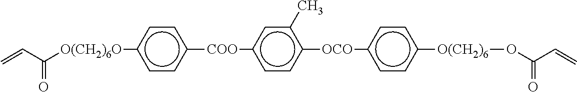 | 5.6 |
| 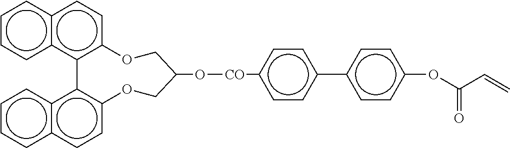 | 3.25 |
| 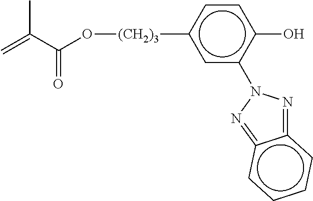 | 2.600 |
| 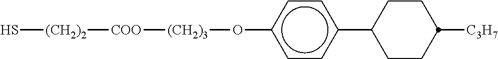 | 2.000 |
| Darocur TPO | 0.240 |
| Irgacure 907 | 4.890 |
| Irganox 1076 | 0.080 |
| Tego Rad 2500 | 0.025 |

### Mixture Example 16: Mixture M-16

| Compound | Conc. |
|---|---|
| 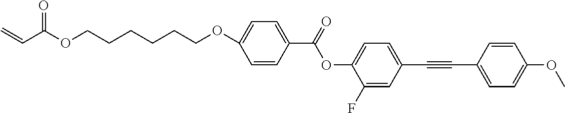 [SMILES: C=CC(=O)OCCCCCCOc1ccc(C(=O)Oc2ccc(C#Cc3ccc(OC)cc3)cc2F)cc1] | 62.475 |
| 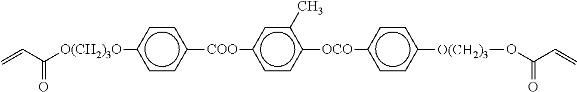 | 32.5 |
| 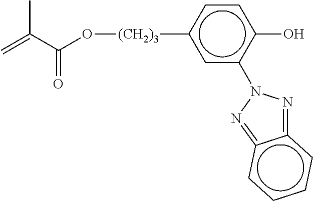 | 1.3 |
| Irgacure 651 | 0.7 |
| Tego Rad 2500 | 0.025 |

### Mixture Example 17: Mixture M-17

| Compound | Conc. |
|---|---|
| 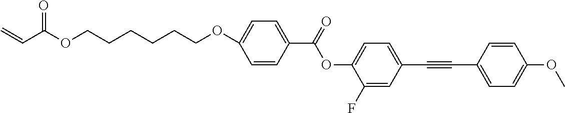 [SMILES: C=CC(=O)OCCCCCCOc1ccc(C(=O)Oc2ccc(C#Cc3ccc(OC)cc3)cc2F)cc1] | 49.75 |
| 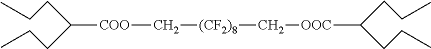 | 1 |
| 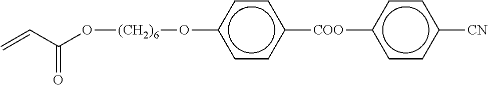 | 10 |
| 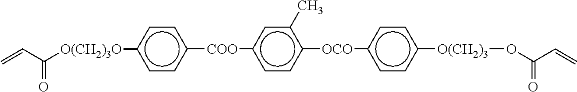 | 22.94 |
| 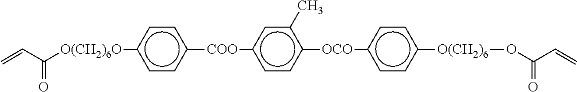 | 5.73 |
| 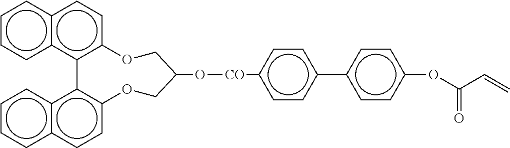 | 2.5 |
| 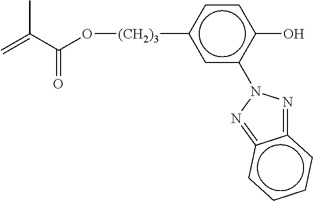 | 1 |
| 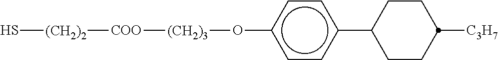 | 2 |
| Irgacure 907 | 5 |
| Irganox 1076 | 0.08 |

### Mixture Example 18: Mixture M-18

| Compound | Conc. |
|---|---|
| 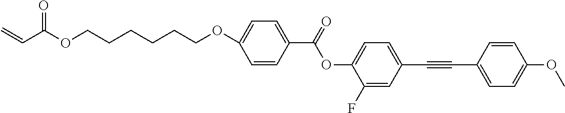 [SMILES: C=CC(=O)OCCCCCCOc1ccc(C(=O)Oc2ccc(C#Cc3ccc(OC)cc3)cc2F)cc1] | 67.745 |
| 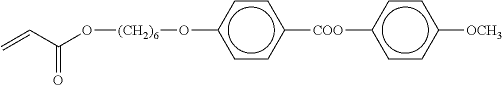 | 5.65 |
| 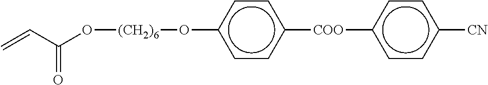 | 5.65 |
| 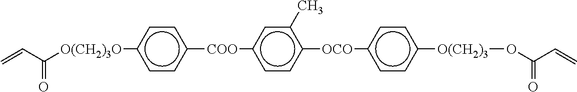 | 8.49 |
| 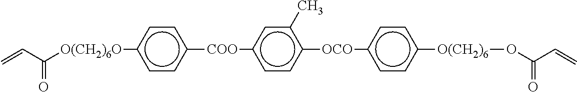 | 2.04 |
| 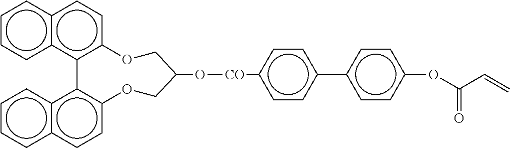 | 3.23 |
| 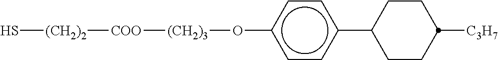 | 1.96 |
| Irgacure 907 | 4.89 |
| Irgacure 819 | 0.24 |
| Irganox 1076 | 0.08 |
| Tego Rad 2500 | 0.025 |

### Mixture Example 19: Mixture M-19

| Compound | Conc. |
|---|---|
| 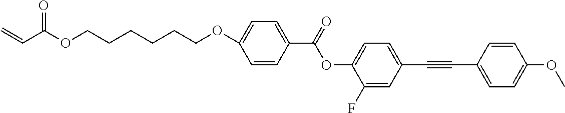 [SMILES: C=CC(=O)OCCCCCCOc1ccc(C(=O)Oc2ccc(C#Cc3ccc(OC)cc3)cc2F)cc1] | 39.01 |
| 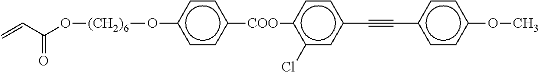 | 10.00 |
| 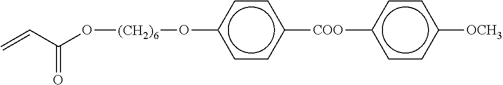 | 9.91 |
| 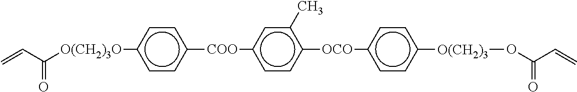 | 22.4 |
| 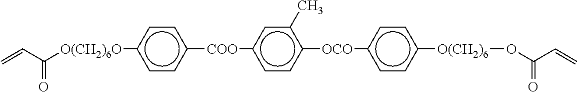 | 5.6 |
| 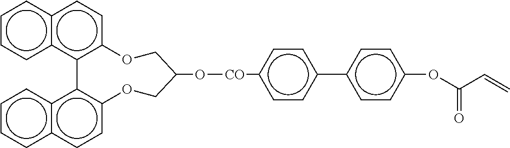 | 3.25 |
| 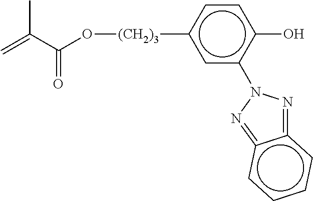 | 2.6 |
| 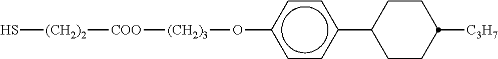 | 2.00 |
| Irgacure 819 | 0.24 |
| Irgacure 907 | 4.89 |
| Irganox 1076 | 0.08 |
| Tego Rad 2500 | 0.02 |

### Mixture Example: Mixture M-20

| Compound | Conc. |
|---|---|
| 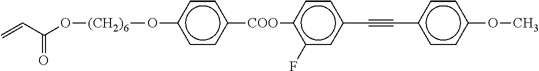 | 66.075 |
| 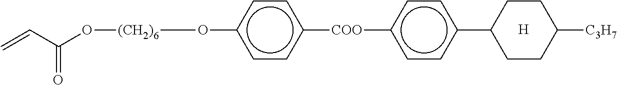 | 11.020 |
| 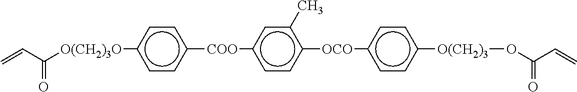 | 8.280 |
| 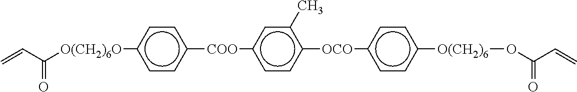 | 1.990 |
| 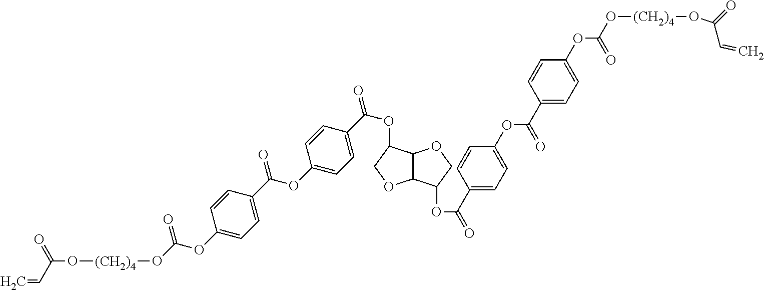 | 5.440 |
| 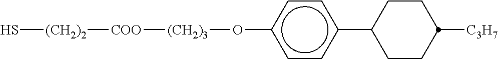 | 1.960 |
| Irgacure 369 | 4.890 |
| Darocur TPO | 0.240 |
| Irganox 1076 | 0.080 |
| Tego Twin 4000 | 0.025 |

The clearing point of comparison mixture C-3 is 140.3° C.

### Mixture Example: Mixture M-21

| Compound | Conc. |
|---|---|
| 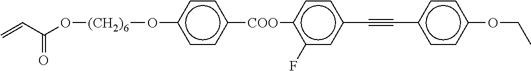 | 15.00 |
| 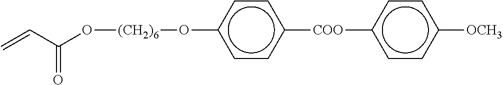 | 9.910 |
| 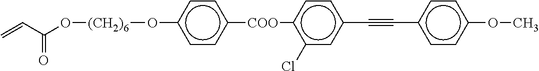 | 34.00 |
| 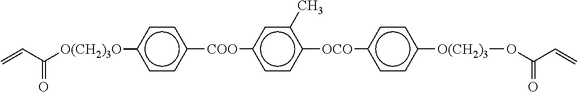 | 22.40 |
| 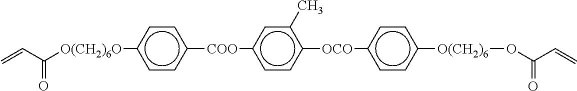 | 5.600 |
| 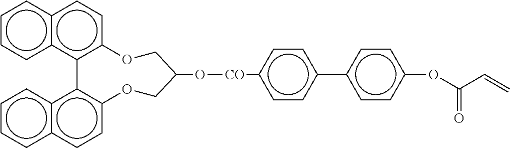 | 3.250 |
| 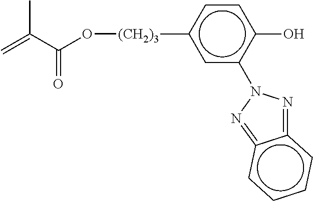 | 2.600 |
| 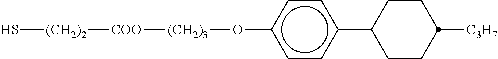 | 2.000 |
| Irgacure 819 | 0.240 |
| Irgacure 907 | 4.890 |
| Irganox 1076 | 0.080 |
| TR2500 | 0.025 |

### Preparation of Polymer Films

The above described mixtures, with the exception of mixtures C-3, C-4 and M-20, are coated using the following process:
Bar coat onto HiFi PET substrate using Meyer bar 10
Anneal in Jisico J-300M forced convection drying oven for 60 sec at 80° C.
UV exposure, high pressure mercury lamp 250-450 nm (Dr. Hoenle), 40 mW/cm² at 40° C. for 30 sec
Post-cure UV exposure, Fusion Light Hammer 6 conveyor lamp, 1 pass at 5 m/min, 100% power (626.5 mJ/cm², 794.8 mW/cm²)

The mixtures C-3, C-4 and M-20 are coated using the following process:
Bar coat onto HiFi PET substrate using Meyer bar 10
Anneal on Stuart SD 300 digital hotplate for 60 sec at 115° C.
UV exposure, Philips 40 W 40-R-25-2.5 TLK lamps 2 mW/cm² at 45° C. for 90 sec
Heat on hotplate at 80° C. for 45 sec
Post-cure UV exposure, DRSE-120QNL Fusion conveyor lamp: 1 pass at 3 m/min 22 cm lamp height 60% power (348.2 mJ/cm², 145.7 mW/cm²), 3 passes at 3 m/min 100% power (2140.7mJ/cm², 313.2 mW/cm²)

### Broadening

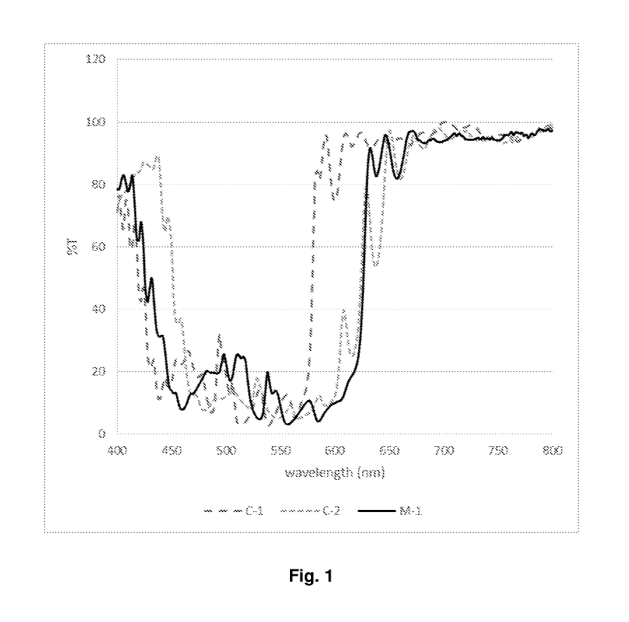

FIG. 1 show the results of broadening studies for mixture M-1 comprising RM-1 of the invention in comparison to mixtures C-1 comprising the compound A of prior art and mixture C-2 comprising the compounds B of prior art.

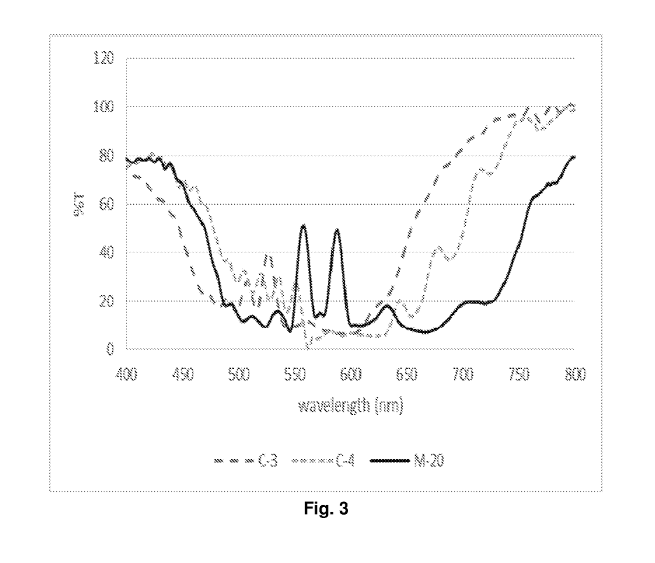

FIG. 3 show the results of broadening studies for mixture M-20 comprising RM-1 of the invention in comparison to mixtures C-3 comprising the compound A of prior art and mixture C-4 comprising the compound B of prior art.

### Solubility

Crystallisation of mixtures C-1, C-2, and M-1 is studied using the following method:
Spincoat 6 drops of solution onto 1 inch rubbed PI glass using SCS G3P-8 spincoater, 1000 rpm, 30 sec
Anneal (in air), 60° C., 60 sec on Stuart hotplate SD160
Place on microscope slide on ‘Olympus MVX10 Macroview’ microscope
Cover with protective shield to prevent dust particles landing on sample
Record image every 30 sec for 12 hrs using Point Grey FlyCap2 software

| Mixture | RM | Crystallisation result after 12 hrs |
|---|---|---|
| C-1 | A | Fail |
| C-2 | B | Fail |
| M-1 | RM-1 | Pass |

1. Compound of formula I, 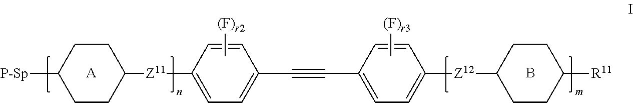
P is a polymerisable group,
Sp is a spacer group or a single bond,
r1, r2 and r3 are independently of each other 0, 1, 2, 3 or 4, with r1+r2+r3≥1
R¹¹ is straight chain or branched alkyl, alkoxy, thioalkyl, alkylcarbonyl, alkoxycarbonyl, alkylcarbonyloxy or alkoxycarbonyloxy with 1 to 15 C atoms which is optionally fluorinated.
A and B denote, in case of multiple occurrence independently of one another, an aromatic or alicyclic group, which optionally contains one or more heteroatoms selected from N, O and S, and is optionally substituted by (F)ᵣ₁,
Z¹¹ and Z¹² denotes, in case of multiple occurrence independently of one another, —O—, —S—, —CO—, —COO—, —OCO—, —S—CO—, —CO—S—, —O—COO—, —CO—NR⁰⁰—, —NR⁰⁰—CO—, —NR⁰⁰—CO—NR⁰⁰, —NR⁰⁰—CO—O—, —O—CO—NR⁰⁰—, —OCH₂—, —CH₂O—, —SCH₂—, —CH₂S—, —CF₂O—, —OCF₂—, —CF₂S—, —SCF₂—, —CH₂CH₂—, —(CH₂)ₙ₁, —CF₂CH₂—, —CH₂CF₂—, —CF₂CF₂—, —CH═N—, —N═CH—, —N═N—, —CH═CR⁰⁰—, —CY¹═CY²—, —C≡C—, —CH═CH—COO—, —OCO—CH═CH— or a single bond,
R⁰⁰ and R⁰⁰⁰ independently of each other denote H or alkyl with 1 to 12 C-atoms,
Y¹ and Y² independently of each other denote H, F, Cl or CN,
n is 1, 2, 3 or 4
m is 0, 1, 2, 3 or 4
n1 is an integer from 1 to 10.

2. The compound according to claim 1, characterized in that it is selected of formula Ia or Ib, 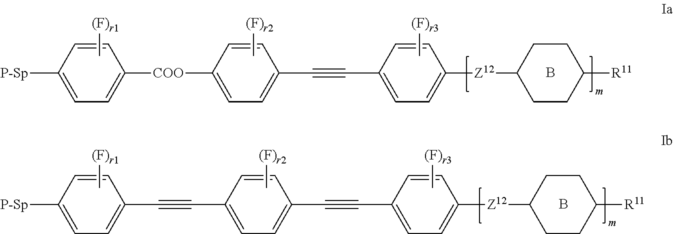
wherein
P is a polymerisable group,
Sp is a spacer group or a single bond,
r1, r2, r3 are independently of each other 0, 1, 2, 3 or 4, with r1+r2+r3≥1, and
R¹¹, Z¹², ring B and m have one of the meanings as given above in claim 1.

3. The compound according to claim 1, characterized in that it is selected from the group of compounds formula I1 to I3 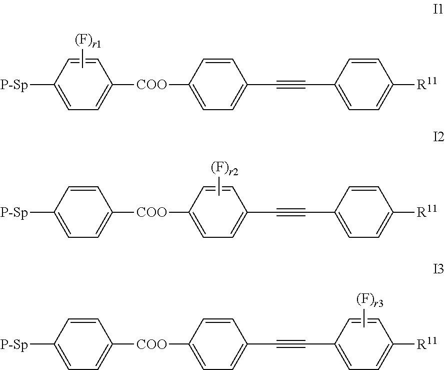
wherein P, Sp, and R¹¹ have one of the meanings as given in claim 1, and r1 to r3 denotes 1, 2, 3, or 4.

4. The compound according to claim 1, wherein P is selected from the group consisting of heptadiene, vinyloxy, acrylate, methacrylate, fluoroacrylate, chloroacrylate, oxetane and epoxide groups.

5. The compound according to claim 1, characterized in that the compound is selected from the group of compounds of formulae I1-A to I1-D, I2-A to I2-D or I3-A to I3-D, 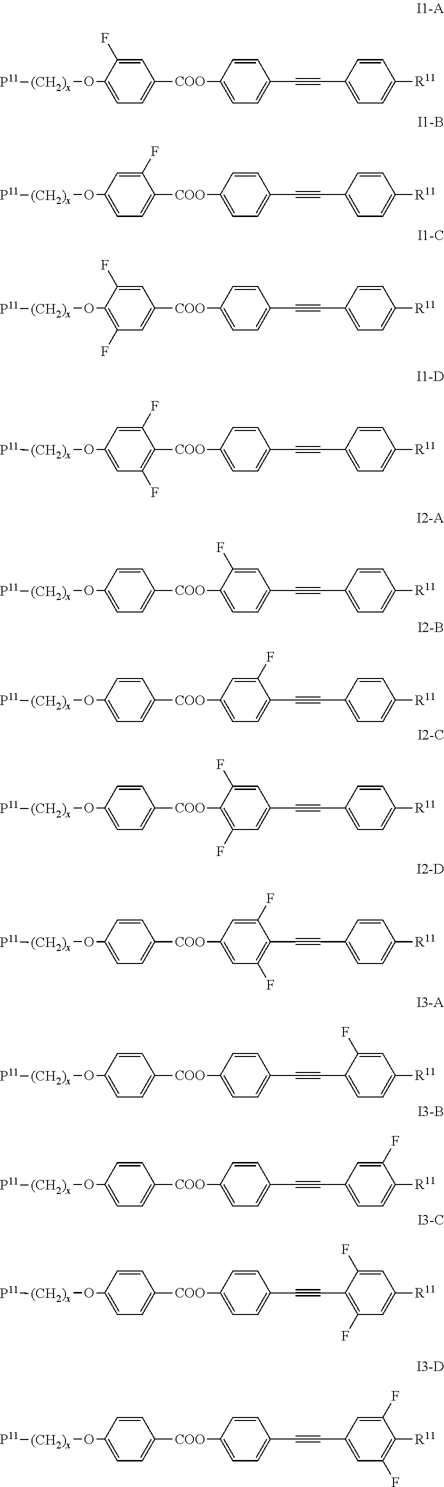
wherein
P¹¹ is selected from the group consisting of vinyloxy, acrylate, methacrylate, fluoroacrylate, chloroacrylate, oxetane and epoxide groups, and very preferably denotes an acrylate, methacrylate or oxetane group, especially an acrylate or methacrylate group, in particular an acrylate group,
x is an integer from 0 to 12, and
R¹¹ has one of the meanings as given above under formula I.

6. The compound according to claim 1, wherein P denotes an acrylate or methacrylate group.

7. The compound according to claim 1, characterized in that the compound is selected from the group of compounds of formulae I2-A1 to I2-D1, 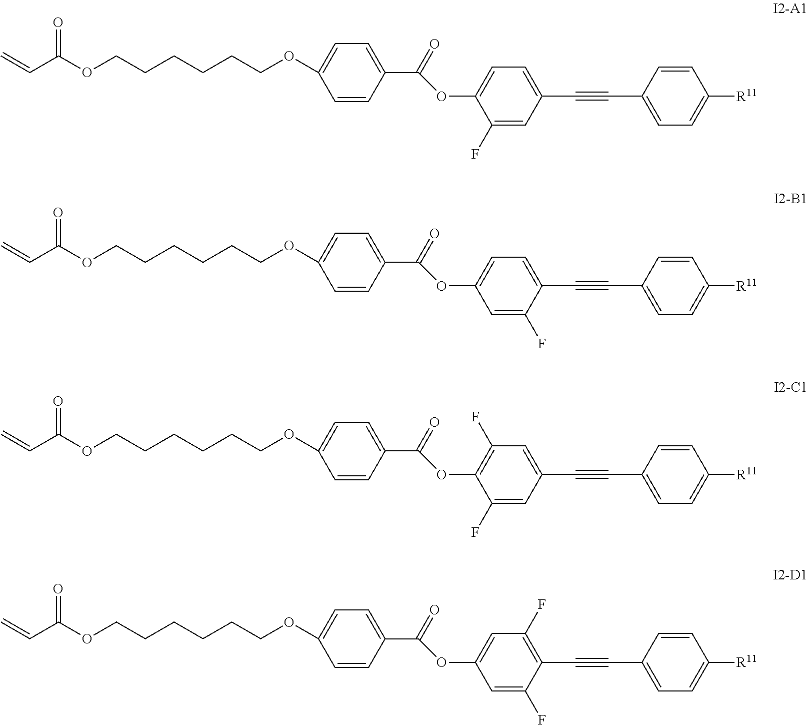
wherein
R¹¹ has one of the meanings as given above under claim 1.

8. The compound according to claim 1, characterized in that R¹¹ denotes alkyl or alkoxy.

9. A mixture comprising two or more reactive mesogens (RMs), at least one of which is a compound of formula I according to claim 1.

10. The mixture according to claim 9, characterized in that it comprises one or more RMs having only one polymerisable functional group, and one or more RMs having two or more polymerisable functional groups.

11. A formulation comprising one or more compounds of formula I according to claim 1, and further comprising one or more solvents and/or additives.

12. A polymer obtained by polymerising a compound of formula I according to claim 1, preferably wherein the RMs are aligned, and preferably at a temperature where the RMs or RM mixture exhibit a liquid crystal phase.

13. Use of the compounds of formula I according to claim 1 in optical, electrooptical or electronic components or devices.

14. An optical, electrooptical or electronic device or a component thereof, comprising a polymer according to claim 12.

15. The component of claim 14, which is selected from optical retardation films, polarizers, compensators, beam splitters, reflective films, alignment layers, colour filters, antistatic protection sheets, electromagnetic interference protection sheets, polarization controlled lenses, IR reflection films, and lenses for light guides, focusing and optical effects.

16. The device of claim 14, which is selected from electrooptical displays, especially LC displays, autostereoscopic 3D displays, organic light emitting diodes (OLEDs), optical data storage devices and windows.

17. The compound according to claim 5, wherein P denotes an acrylate or methacrylate group
P¹¹ is selected from the group consisting of vinyloxy, acrylate, methacrylate, fluoroacrylate, chloroacrylate, oxetane and epoxide groups, and very preferably denotes an acrylate, methacrylate or oxetane group, especially an acrylate or methacrylate group, in particular an acrylate group.

18. An optical, electrooptical or electronic device or component thereof, comprising a mixture according to claim 9.
